(19) United States
(12) Patent Application Publication
(10) Pub. No.: US 2018/0140744 A1
(43) Pub. Date: May 24, 2018

(54) POROUS MATRIX WITH INCORPORATED CELLS

(71) Applicant: SKIN TISSUE ENGINEERING PTY LTD, Unley, South Australia (AU)

(72) Inventor: John GREENWOOD, UNLEY, SOUTH AUSTRALIA (AU)

(73) Assignee: SKIN TISSUE ENGINEERING PTY LTD, Unley, South Australia (AU)

(21) Appl. No.: 15/571,977

(22) PCT Filed: Oct. 19, 2016
(86) PCT No.: PCT/AU2016/000360

§ 371 Date: Nov. 6, 2017

(30) Foreign Application Priority Data
Oct. 19, 2015 (AU) 2015904282

(51) Int. Cl. A61L 27/38; A61L 27/52; A61L 27/36; C12N 5/00; A61L 27/56; A61L 27/58; A61L 27/60; C12M 1/12; G09B 23/30; A61L 27/44

(52) U.S. Cl. CPC A61L 27/3813; A61L 27/52; A61L 27/3687; C12N 5/0062; A61L 27/56; A61L 27/58; C12N 2533/56; C12M 25/14; G09B 23/306; A61L 27/44; A61L 2430/34; A61L 2430/40; C08L 2203/02; A61L 27/60

(57) ABSTRACT

A method of incorporating cells in a porous matrix, the method comprising incorporating the cells in the porous matrix by forming a gel comprising the cells in situ in the porous matrix from a gellable medium, wherein the formation of the gel from the gellable medium comprises a time period of 10 minutes or less after contact of the gellable medium with the cells, thereby incorporating the cells in the porous matrix. The incorporated cells can then be used, for example, to produce a cultured skin product or deliver specific cells to a subject in need thereof.

### FIELD

The present disclosure relates to methods for incorporating cells into a porous matrix, porous matrices with incorporated cells and uses of porous matrices with incorporated cells.

### BACKGROUND

There are a variety of situations where introducing cells into a subject can be used for therapeutic purposes. For example, autologous chondrocyte implantation can be used to repair cartilage defects. The introduction of stem cells or progenitor cells into subjects is also a new avenue for the treatment of many diseases and conditions.

In some cases, a carrier or scaffold is used to assist with the introduction of the cells, and/or assist with structural integrity or the formation of new supporting tissue. For example, implants using a scaffold with chondrocytes have therapeutic potential for the repair of cartilage.

The seeding of skin cells in a carrier or scaffold to form skin is also an avenue currently being explored for wound treatment. However, these substitute skins suffer from a variety of disadvantages, not least the cost of production.

Another obstacle to the use of carriers or scaffolds is how to incorporate the cells into the carrier or scaffold. In some cases, the cells need to be incorporated in the carrier or scaffold in a manner that is compatible with the structural and/or positional constraints on the cells when they are introduced into the subject.

A further consideration is also applicable to the introduction of autologous cells. In this case, there is often a need to provide sufficient cell numbers in a time to meet the therapeutic window available. This is particularly the case for large burns.

Accordingly, for a variety of reasons there is a need for improved methods of introducing cells into subjects, and in particular improved methods for introducing cells into a subject using a carrier or scaffold.

### SUMMARY

Certain embodiments of the present disclosure provide a method of incorporating cells in a porous matrix, the method comprising incorporating the cells in the porous matrix by forming a gel comprising the cells in situ in the porous matrix from a gellable medium, wherein the formation of the gel from the gellable medium comprises a time period of 10 minutes or less after contact of the gellable medium with the cells, thereby incorporating the cells in the porous matrix.

Certain embodiments of the present disclosure provide a method of incorporating cells in a porous matrix, the method comprising:
applying a gellable medium to the porous matrix; and
applying cells and a gelling agent to the porous matrix with applied gellable medium to form a gel comprising the cells, wherein the formation of the gel from the gellable medium comprises a time period of 10 minutes or less after contact of the gellable medium with the cells;
thereby incorporating the cells in the porous matrix.

Certain embodiments of the present disclosure provide a method of incorporating cells in a porous matrix, the method comprising:
applying plasma and/or a derivative thereof to the porous matrix; and
applying cells and thrombin to the porous matrix with applied plasma to form a gel comprising the cells, wherein the formation of the gel comprises a time period of 10 minutes or less after contact of the plasma with the cells;
thereby incorporating the cells in the porous matrix.

Certain embodiments of the present disclosure provide a porous matrix comprising a gel comprising cells, the gel comprising the cells being formed in situ from a gellable medium, wherein the formation of the gel from the gellable medium comprises a time period of 10 minutes or less after contact of the gellable medium with the cells.

Certain embodiments of the present disclosure provide a porous matrix comprising a gel comprising cells, the cells in the gel being substantially uniformly distributed in the gel.

Certain embodiments of the present disclosure provide a method of producing a product for forming skin, the method comprising:
incorporating fibroblast cells and/or keratinocyte cells, and/or progenitors thereof, in a porous matrix by forming one or more gels comprising the cells in situ in the porous matrix from a gellable medium, wherein the formation of the one or more gels from the gellable medium comprises a time period of 10 minutes or less after contact of the gellable medium with the cells; and
incubating the porous matrix with incorporated fibroblast cells and/or keratinocyte cells, and/or progenitors thereof, to expand numbers of cells;
thereby producing a product for forming skin.

Certain embodiments of the present disclosure provide a method of producing a product for forming skin, the method comprising:
applying plasma to a biodegradable porous matrix;
applying fibroblast cells, and/or a progenitor thereof, and thrombin to the biodegradable porous matrix with applied plasma to form a gel comprising fibroblast cells incorporated in the porous matrix;
incubating the porous matrix with incorporated fibroblast cells;
applying keratinocyte cells, and/or a progenitor thereof, and thrombin to the porous matrix with incorporated fibroblast cells to form a gel comprising keratinocyte cells incorporated in the porous matrix; and
incubating the porous matrix with incorporated fibroblast cells and keratinocyte cells;
wherein the formation of the said gels comprises a time period of 10 minutes or less after contact of the plasma with the cells;
thereby producing a product for forming skin.

Certain embodiments of the present disclosure provide a method of producing a product for forming skin in a subject, the method comprising:
applying autologous plasma, and/or a derivative thereof, to a biodegradable porous matrix;
applying autologous fibroblast cells, and/or a progenitor thereof, and thrombin to the biodegradable porous matrix with applied plasma to form a gel comprising fibroblast cells incorporated in the porous matrix;
incubating the porous matrix with incorporated fibroblast cells;
applying autologous keratinocyte cells, and/or a progenitor thereof, and thrombin to the porous matrix with incorporated fibroblast cells to form a gel comprising keratinocyte cells incorporated in the porous matrix; and
incubating the porous matrix with incorporated fibroblast cells and keratinocyte cells;
wherein the formation of the said gels comprises a time period of 10 minutes or less after contact of the plasma with the cells;
thereby producing a product for forming skin in a subject.

Certain embodiments of the present disclosure provide a method of expanding autologous cells for introduction into a subject, the method comprising:
incorporating autologous cells in a porous matrix by forming a gel comprising the cells in situ in the porous matrix from a gellable medium, wherein the formation of the gel from the gellable medium comprises a time period of 10 minutes or less after contact of the gellable medium with the cells; and
incubating the porous matrix to expand the cells.

Certain embodiments of the present disclosure provide a system for producing cultured skin, the system comprising:
(i) a product comprising a porous matrix with incorporated cells, the cells being incorporated in the porous matrix by forming a gel comprising the cells in situ in the porous matrix from a gellable medium, wherein the formation of the gel from the gettable medium comprises a time period of 10 minutes or less after contact of the gellable medium with the cells; and
(ii) a bioreactor for culturing the product to produce cultured skin.

Other embodiments as described herein.

### BRIEF DESCRIPTION OF THE FIGURES

Exemplary embodiments will be better understood and appreciated in conjunction with the following detailed description of example embodiments taken together with the accompanying figures. It is to be understood that the following description of the figures is for the purpose of describing example embodiments only and is not intended to be limiting with respect to this disclosure.

FIG. 1 shows clinical and histological progression of the integrated BTM from pig 2 post composite application. A, Day 7. B, Day 10. C, Day 14. D, Higher magnification of the CCS “eschar” removed to reveal keratinized epithelium below. E, Central punch biopsy day 7. F, Day 10. G, Day 14. H, Day 14, showing a section where no CCS was visible.

FIG. 2 shows clinical and histological progression of the other integrated BTM from pig 2 post composite application. A, Day 3. B, Day 7. C, Day 10. D, Day 14. Histology of 1-mm thick CCS applied onto integrated BTM at day 28. E, H&E section with CCS “take” with matrix integration. F, PAS section demonstrating basement membrane formation and matrix integration at day 7 post application. G, BovK staining for keratin in composite epidermis. Ks, keratinocytes; BKs, basal keratinocytes; Fbs, Fibroblasts/collagen; BM, basement membrane; SF, subcutaneous fat.

FIG. 3 shows representative micrographs of the CCS. A, Spindle-shaped fibroblasts in the polymer foam scaffold. B, A continuous sheet (monolayer) of keratinocytes, detached from the polymer matrix, staining immunopositively for cytokeratin, with a central immunonegative area containing fibroblasts with elongated nuclei. Keratinocytes at higher power (inset). C, Immunostaining of keratin with BovK, green and fibroblast nuclei stained red with propidium iodide. D, Stained as per C but higher power showing detachment of cells and clumping of keratin-positive cells (green).

FIG. 4 shows a temporal series of clinical and histological appearance of the cultured composite skin applied alone to pig 1, showing the composite as a delivery vehicle for cells. A, Day 3. B, Day 7. C, Day 10. D, Day 14. E, At day 10, the CCS appears as an overlying “stiff carapace” and when lifted. F, An epithelium is present below. G, Higher power of the reepithelialisation extending to the center of the wound. H, H&E section confirming a delivery vehicle, as no polymer matrix visible. I, Positively-stained (brown) keratinized epithelium with BovK. J, PAS staining showing basement membrane formation day 10 post application. Ks, keratinocytes; BKs, basal keratinocytes; Fbs, fibroblasts/collagen.

FIG. 5 shows a temporal series of cultured composite skin alone from pig 2 showing composite “take.” A, Day 3 post application. B, Integration at day 7. C, Central epithelialization covering ˜70% of wound on day 10. D, Virtually 100% healed with robust epithelium by day 14 post application. E and F, A punch biopsy taken centrally from the wound on day 10, with surrounding epithelium. G, Histology from the punch biopsy shows composite “take,” and a well-developed epithelium. H, H&E section from day 14 post application displaying composite “take.”

FIG. 6 shows graphical representation for the optimization of the biodegradable temporizing matrix (BTM) seal. Wound area over time with different BTM seal properties and bonding. Error bars are the SEM.

FIG. 7 shows serial progression of an optimized biodegradable temporizing matrix before seal delamination/removal. A. Day 0, (B) day 3, (C) day 7, (D) day 10, (E) day 14 to (F) day 21 after surgery. Original wound area=65 cm²; day 21 predelamination wound area=63 cm².

FIG. 8 shows cultured composite skin in vitro before application. A. Fibroblasts (Fbs) stained green (calcein), filling the polymer pore. B. Co-cultured fibroblasts and keratinocytes (Ks) lining the polymer wall (polymer scaffold stained red). C. Hematoxylin and eosin horizontal section demonstrates a single pore with keratinocytes bordering the polymer edge with central fibroblasts. D. Immunopositive staining (brown) for keratin (BovK).

FIG. 9 shows cultured composite skin (CCS) integration and “take” at (A) day 7 and (B) day 10 after application. (i) Central punch biopsy location; (ii) hematoxylin and eosin histology of punch biopsy sites; (iii) high magnification of CCS epithelium; (iv) periodic acid-Schiff section showing basement membrane (BM) development. BTM, biodegradable temporizing matrix.

FIG. 10 shows cultured composite skin (CCS) delivery vehicle after application at (A) day 7 and (B) day 10. (i) Central location of punch biopsy sites; (Aii) hematoxylin and eosin histology of punch biopsy sites; (Bii) high magnification of reepithelialization; (iii) high magnification of CCS epithelium; (iv) periodic acid-Schiff section showing basement membrane development (BM). BTM, biodegradable temporizing matrix.

FIG. 11 shows cultured composite skin (CCS) “take” on a freshly created deep wound at (A) day 21 (0), (B) day 24 (3), (C) day 28 (7), and (D) day 31 (10), (E) enlargement showing foci epithelium deposited from the CCS, (F) hematoxylin and eosin (H&E) histology of punch biopsy sites, (G) H&E (×10) staining of integrated CCS enveloped with epithelium at day 10, and (H) periodic acid-Schiff staining showing basement membrane (BM) development.

FIG. 12 shows wound contraction comparison, starting at day 21 when (A) the cultured composite skin is applied to the integrated biodegradable temporizing matrix and (B) the fresh control wound is created and allowed to heal by secondary intention, (i) day 21 (0); (ii) day 31 (10); (iii) day 38 (17); (iv) day 42 (21); (v) day 52 (31) after wound creation.

FIG. 13 shows integrated biodegradable temporizing matrix (BTM) and delamination of new seal. A. Seal being delaminated at day 28, (B) highly vascularized “neodermis” before dermabrasion; (C) hematoxylin and eosin section showing seal attachment and minimal ingrowth between the seal and the 2-mm matrix.

### DETAILED DESCRIPTION

The present disclosure relates to methods for incorporating cells into a porous matrix, porous matrices having incorporated cells and uses of the porous matrices with incorporated cells.

Certain embodiments of the present disclosure are directed to methods, products and systems. For example, some of the advantages of the embodiments disclosed herein include one or more of the following: new methods for incorporating cells into a porous matrix; methods for incorporating cells in a porous matrix which assist with the cells forming new tissue; a matrix for incorporating cells that is readily sterilisable; a method for incorporating cells into a matrix whereby the matrix can be biodegradable, if so desired; methods for incorporating cells in a porous matrix so that the cells are distributed in a manner which assists with the formation of new tissue; new methods of incorporating cells in a porous matrix by forming a gel with cells in situ in the porous matrix; new products for introducing or delivering cells to a subject; new products for the production of cultured skin; new products for the treatment of wounds or burns; new methods and products for introducing or delivering autologous cells; methods and products for delivering cells that can be produced with reduced cost; new methods for expanding cells using a porous matrix with the cells incorporated therein; to provide methods for treating wounds or burns with improvements to patient care and/or treatment costs; to provide one or more advantages; or to provide a commercial alternative. Other advantages of certain embodiments of the present disclosure are also disclosed herein.

The present disclosure is based, at least in part, from the recognition that cells may be incorporated in a porous matrix by using a gel with cells in the porous matrix formed in situ, and that the rate of formation and/or concentration of the gel imparts important advantages as to how the cells proliferate and are located and/or arranged in the porous matrix and/or how new tissue can be formed.

In particular, the time of gel formation has effects on how the cells proliferate and/or are located and/or arranged in the porous matrix upon gel formation in situ and/or how new tissue can be formed from cells incorporated in this way. For example, a time of gel formation as described herein provides a gel formed in situ that prevents cells from settling prior to gel formation. In addition, the gel formed in this manner provides a rigidity that assists with the formation of new tissue being formed from the incorporated cells without the gel being too stiff and/or solid. The formation of a gel in situ also assists with the porous matrix attaching to the culture vessel and therefore not floating when media is added.

Certain embodiments of the present disclosure provide a method of incorporating cells in a porous matrix.

Certain embodiments of the present disclosure provide a method of incorporating cells in a porous matrix, the method comprising incorporating the cells in the porous matrix by forming a gel comprising the cells in situ in the porous matrix from a gellable medium, wherein the formation of the gel from the gellable medium comprises a time period of 10 minutes or less after contact of the gellable medium with the cells, thereby incorporating the cells in the porous matrix.

The term “porous matrix” as used herein refers to a solid or a semi-solid substrate, carrier or scaffold having openings or apertures in the matrix which allow a cell/cells partially or completely to occupy the openings and/or apertures, and/or openings or apertures through which a cell, or a cluster of cells may pass/reside. Examples of matrices include a solid matrix with pores, a fibrous matrix, a mat, a mesh, a sponge, or a foam. Other types of porous matrix are contemplated.

Methods for determining the time of gel formation are known in the art. For example, using a plasma or a fibrin gel, the time of gel formation may be determined by measuring a change of absorbance at 550 nm of the gellable medium as a function of reaction time, using a UV-VIS spectrophotometer, and determining the time at which the maximum value for absorbance occurs.

In certain embodiments, the formation of the gel comprises a time period of 9 minutes or less, 8 minutes or less, 7 minutes or less, 6 minutes or less, 5 minutes or less, 4 minutes or less, 3 minutes or less, 2 minutes or less, 1 minute or less or a time period about one of the aforementioned time periods.

In certain embodiments, the formation of the gel comprises a time period for formation of the gel after contact of the gellable medium with the cells of 10 to 300 seconds, 20 to 300 seconds, 25 to 300 seconds, 30 to 300 seconds, 45 to 300 seconds, 50 to 300 seconds, 60 to 300 seconds, 90 to 300 seconds, 120 to 300 seconds, 150 to 300 seconds, 180 to 300 seconds, 210 to 300 seconds, 240 to 300 seconds, 270 to 300 seconds, 10 to 270 seconds, 20 to 270 seconds, 25 to 270 seconds, 30 to 270 seconds, 45 to 270 seconds, 50 to 270 seconds, 60 to 270 seconds, 90 to 270 seconds, 120 to 270 seconds, 150 to 270 seconds, 180 to 270 seconds, 210 to 270 seconds, 240 to 270 seconds, 10 to 240 seconds, 20 to 240 seconds, 25 to 240 seconds, 30 to 240 seconds, 45 to 240 seconds, 50 to 240 seconds, 60 to 240 seconds, 90 to 240 seconds, 120 to 240 seconds, 150 to 240 seconds, 180 to 240 seconds, 210 to 240 seconds, 10 to 210 seconds, 20 to 210 seconds, 25 to 210 seconds, 30 to 210 seconds, 45 to 210 seconds, 50 to 210 seconds, 60 to 210 seconds, 90 to 210 seconds, 120 to 210 seconds, 150 to 210 seconds, 180 to 210 seconds, 10 to 180 seconds, 20 to 180 seconds, 25 to 180 seconds, 30 to 180 seconds, 45 to 180 seconds, 50 to 180 seconds, 60 to 180 seconds, 90 to 180 seconds, 120 to 180 seconds, 150 to 180 seconds, 10 to 150 seconds, 20 to 150 seconds, 25 to 150 seconds, 30 to 150 seconds, 45 to 150 seconds, 50 to 150 seconds, 60 to 150 seconds, 90 to 150 seconds, 120 to 150 seconds, 10 to 120 seconds, 20 to 120 seconds, 25 to 120 seconds, 30 to 120 seconds, 45 to 120 seconds, 50 to 120 seconds, 60 to 120 seconds, 90 to 120 seconds, 10 to 90 seconds, 20 to 90 seconds, 25 to 90 seconds, 30 to 90 seconds, 45 to 90 seconds, 50 to 90 seconds, 60 to 90 seconds, 10 to 60 seconds, 20 to 60 seconds, 25 to 60 seconds, 30 to 60 seconds, 45 to 60 seconds, 50 to 60 seconds, 10 to 50 seconds, 20 to 50 seconds, 25 to 50 seconds, 30 to 50 seconds, 45 to 50 seconds, 10 to 45 seconds, 20 to 45 seconds, 25 to 50 seconds, 30 to 45 seconds, 10 to 30 seconds, 20 to 30 seconds, 25 to 30 seconds, 10 to 25 seconds, 20 to 25 seconds, 10 to 20 seconds, or a time period about one of the aforementioned time periods. Other time periods are contemplated.

The term “about” means an acceptable error for a particular value, which depends in part on how the value is measured or determined. When the term “about” is applied to a recited range or value it denotes an approximation within the deviation in the range or value known or expected in the art from the measurements method. For removal of doubt, it shall be understood that any range stated herein that does not specifically recite the term “about” in conjunction with the range or any value within the stated range inherently includes such term to encompass the approximation within the deviation noted above.

In certain embodiments, the formation of the gel comprises a time period of 10 to 300 seconds after contact of the gellable medium with the cells.

In certain embodiments, the formation of the gel comprises a time period of 10 to 50 seconds after contact of the gellable medium with the cells. In certain embodiments, the formation of the gel comprises a time period of 25 to 50 seconds after contact of the gellable medium with the cells.

In certain embodiments, the formation of the gel comprises a time period of 10 to 300 seconds after contact of the gellable medium with the cells. In certain embodiments, the formation of the gel comprises a time period of one of 10 to 50, 20 to 50, 25 to 50, 30 to 50, 10 to 30, or 20 to 30 seconds after contact of the gellable medium with the cells.

In certain embodiments, the cells comprise skin cells. In certain embodiments, the cells comprise one or more of fibroblast cells and/or a precursor thereof, keratinocyte cells and/or a precursor thereof, hair follicle cells and/or a precursor thereof, sweat gland cells and/or a precursor thereof, sebaceous gland cells and/or a precursor thereof, and melanocyte cells and/or a precursor thereof.

In certain embodiments, the cells comprise cells that produce a paracrine factor, such as a growth factor.

In certain embodiments, the cells comprise cells for forming part of a tissue or organ, such as skin. In certain embodiments, the cells comprise cells for assisting with forming a structural component.

In certain embodiments, the cells comprise stem cells, or a progenitor of a desired cell type.

In certain embodiments, the cells comprise one cell type. In certain embodiments, the cells comprise different cell types. In certain embodiments, the cells comprise more than one cell type. In certain embodiments, the cells comprise fibroblasts and keratinocytes.

Methods for producing cells for incorporation in the porous matrix are known in the art. Sources of cells for incorporation in the porous matrix are known in the art. For example, sources of fibroblast cells and keratinocyte cells include skin biopsies. Methods for characterising cells for their suitability for incorporation are known in the art.

In certain embodiments, the cells for incorporation comprise autologous cells derived from a subject.

In this regard, the term “subject” as used herein refers to a human or animal subject.

In certain embodiments, the subject is a mammalian subject, a livestock animal (such as a horse, a cow, a sheep, a goat, a pig), a domestic animal (such as a dog or a cat) and other types of animals such as primates, rabbits, rats, mice, birds and laboratory animals. Other types of animals are contemplated. Veterinary applications of the present disclosure are contemplated.

In certain embodiments, the subject is a human subject.

In certain embodiments, the subject is suffering from, or susceptible to, a disease, condition or state that would benefit from the delivery of cells to the subject.

For example, the method may be used in treatment regimes that are beneficial for wound healing, such as a wound that occurs during surgery or a burn wound. Examples of wounds include acute wounds, chronic wounds, diabetic wounds and diabetic ulcers.

Methods for producing a porous matrix are known in the art.

For example, a fibrous porous matrix may be produced by an extrusion process (see for example Greenwood et al. (2010) “Evaluation of NovoSorb™ novel biodegradable polymer for the generation of a dermal matrix Part 1: In-vitro Studies” Wound Practice and Research 18(1): 14-22) or a process such as electrospinning (see for example Brown et al. (2014) Materials Science and Engineering: C, 2014, 45, 698-708).

In certain embodiments, the porous matrix comprises an average pore size of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Methods for determining pore size are known in the art. In certain embodiments, the pore size comprises the size of the largest cross sectional diameter of a pore.

In certain embodiments, the porous matrix comprises an average pore size in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 gm, 500 μm to 900 μm, 600 μm to 900 μm, 700 μm to 900 μm, 800 μm to 900 μm, 100 μm to 800 μm, 200 μm to 800 μm,300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm.

In certain embodiments, the porous matrix comprises an average pore size of greater than 100 μm to 1000 μm. In certain embodiments, the porous matrix comprises an average pore size of greater than 200 μm to 500 μm.

In certain embodiments, the porous matrix is biodegradable in vivo. In certain embodiments, the porous matrix comprises a biodegradable polymer. In certain embodiments, the biodegradable polymer has a half life in vivo (for example as measured by the mass retained) of 30 to 120 days.

In certain embodiments, the porous matrix is non-biodegradable in vivo. In certain embodiments, the porous matrix comprises a non-biodegradable polymer.

Examples of matrices are as described herein. In certain embodiments, the porous matrix includes a fibrous matrix.

Porous matrices may be produced by a method known in the art or obtained commercially.

In certain embodiments, the porous matrix comprises a fibrous matrix, a sheet, a mesh, a mat, a woven matrix, a cloth, a foam and/or a sponge. Other types of matrix are contemplated.

In certain embodiment, the porous matrix comprises a synthetic matrix. In certain embodiments, the porous matrix comprises a natural matrix or a matrix derived from a natural product, such as a collagen or a chitosan.

In certain embodiments, the porous scaffold is sterilisable. In certain embodiments, the porous scaffold is shelf stable.

In certain embodiments, the porous matrix comprises a fibrous porous matrix.

In certain embodiments, the fibrous porous matrix comprises fibres with a diameter in the range from 50 μm to 200 μm.

In certain embodiments, the fibrous porous matrix comprises fibres with a diameter in the range from 60 μm to 100 μm.

In certain embodiments, the porous matrix is in the form of a sheet or a mat.

In certain embodiments, the sheet comprises a thickness of 1 mm or greater, 2 mm or greater or 3 mm or greater. In certain embodiments, the sheet comprises a thickness of at least 1 mm, at least 2 mm, or at least 3 mm. Other sizes are contemplated.

In certain embodiments, the sheet comprises a thickness in the range from 1 mm to 3 mm.

In certain embodiments, the cells applied to the porous matrix comprise 1×10⁴ or greater cells per cm² of the porous matrix. In certain embodiments, the cells applied to the porous matrix comprise 4×10⁴ or greater cells per cm² of the porous matrix. In certain embodiments, the cells applied to the porous matrix comprise 1×10⁵ or greater cells per cm² of the porous matrix. Other cell densities are contemplated.

In certain embodiments, the cells applied to the porous matrix comprise 1×10³ to 1×10⁶ cells per cm² of the porous matrix. In certain embodiments, the cells applied to the porous matrix comprise 1×10⁴ to 1×10⁵ cells per cm² of the porous matrix.

In certain embodiments, the porous matrix comprises one or more of a polyurethane, a collagen, a glycosaminoglycan, a collagen-GAG co-polymer and/or mixture, a mucopolysaccharide, a poly(alpha-hydroxy acid), a poly(lactic acid) and/or a structural isomer thereof, a polyglycolic acid and/or a structural isomer thereof, a polylactic-polyglycolic polymer, a chitosan, and a polycaprolactone. Other materials are contemplated.

In certain embodiments, the porous matrix comprises a polyurethane. Polyurethane polymers are described, for example, in international patent publications WO 2008/014561; WO 2009/043099, WO 2005/089778; WO/2004/009227 and WO/2005/085312.

In certain embodiments, the porous matrix comprises a biodegradable polyurethane.

For example, a polyurethane matrix may be produced as described in Greenwood et al. (2010) “Evaluation of NovoSorb™ novel biodegradable polymer for the generation of a dermal matrix Part 1: In-vitro Studies” Wound Practice and Research 18(1): 14-22.

In certain embodiments, the gellable medium forms a gel upon exposure of the gellable medium to a condition, treatment and/or agent. Examples include a change in temperature, UV irradiation, or an enzyme.

Examples of gellable media include plasma, and/or a gellable derivative thereof, plasma and culture medium, plasma and a supplement, plasma and serum, plasma and culture medium and serum, a fibrinogen, a polysaccharide (such as an alginate, an agarose, a chitosan, a pectate), a polymer (such as a polyvinyl alcohol polymer), and a protein (such as gelatin, collagen).

In certain embodiments, the gellable medium comprises an autologous gellable medium.

In certain embodiments, the gellable medium comprises plasma and/or a gellable derivative thereof. In certain embodiments, the derivative of plasma comprises a component, extract, or fraction of plasma. In certain embodiments, the derivative of plasma comprises a component comprising fibrinogen. In certain embodiments, the gellable medium comprises plasma and/or culture medium and/or serum.

In certain embodiments, the method comprises applying the cells and the gellable medium to the porous matrix at the same time. For example, cells for incorporation may be resuspended in the gellable medium, such as plasma.

In certain embodiments, the method comprises applying the gellable medium to the porous matrix and subsequently applying the cells to the porous matrix. For example, plasma may be applied to the porous matrix and a solution containing cells for incorporation added to the plasma applied to the porous matrix.

In certain embodiments, the method comprises use of a gelling agent to form a gel from the gellable medium.

In certain embodiments, the gellable medium comprises plasma and/or a derivative thereof and the gelling agent comprises a fibrinogen-clotting serine protease, such as a thrombin. In certain embodiments, the gelling agent comprises a thrombin.

In certain embodiments, the gelling agent comprises culture medium and/or serum, and/or derivatives thereof.

In certain embodiments, the gelling agent comprises an autologous gelling agent. In certain embodiments, the gelling agent comprises an autologous thrombin.

In certain embodiments, the use of the thrombin comprises a plasma/thrombin ratio of 60:40 to 85:15. In certain embodiments, the use of the thrombin comprises a plasma/thrombin ratio of 75:25 to 85:15. In certain embodiments, the use of the thrombin comprises a plasma/thrombin ratio of 60:40 to 82:18. In certain embodiments, the use of the thrombin comprises a plasma/thrombin ratio of 75:25 to 82:18.

In this regard, the range of thrombin used in conjunction with plasma (and/or a gellable derivative thereof) has been selected so as to assist with how the cells proliferate and/or are located and/or arranged in the porous matrix upon gel formation in situ and/or how new tissue can be formed from cells incorporated in this way. For example, the concentration of thrombin provides a gel formed in situ that prevents cells from settling prior to gel formation. In addition, the gel formed in this manner provides a rigidity that assists with the formation of new tissue being formed from the incorporated cells without the gel being too stiff and/or solid. The formation of a gel in situ also assists with the porous matrix attaching to the culture vessel and therefore not floating when media is added.

In certain embodiments, the gelling agent comprises endogenous thrombin. In certain embodiments, the gelling agent comprises exogenous thrombin.

Certain embodiments of the present disclosure provide a method of incorporating cells in a porous matrix, the method comprising incorporating the cells in the porous matrix by forming a gel comprising the cells in situ in the porous matrix from a gellable medium, wherein the formation of the gel from the gellable medium comprises a time period of 10 minutes or less after contact of the gellable medium with the cells, thereby incorporating the cells in the porous matrix.

In certain embodiments, the method comprises incorporating different types of cells at the same time, for example, incorporating fibroblasts and keratinocytes at the same time. In certain embodiments, the method comprises forming the gel comprising different cell types in situ in the porous matrix. In certain embodiments, the method comprises applying different types of cells at the same time.

In certain embodiments, the method comprises one or more rounds of gel formation incorporating cells in the porous matrix. For example, a first layer of cells may be incorporated in the porous matrix, and after the gel has set (and optionally cultured to expand the cells), a layer of further cells may be incorporated in the porous matrix by formation of a gel in situ carrying the further cells.

In certain embodiments, the method comprises one or more rounds of applying fibroblast cells, and/or a progenitor thereof, to form a gel comprising fibroblast cells, and/or a progenitor thereof, and one or more rounds of applying keratinocyte cells, and/or a progenitor thereof, to form a gel comprising keratinocytes cells.

In certain embodiments, the keratinocyte cells are applied subsequent to the applying of fibroblast cells.

In this manner, for example, a composite cultured skin product may be formed from fibroblasts and/or keratinocytes.

In certain embodiments, the method further comprises incubating the cells incorporated in the porous matrix to expand cell numbers. For example, the porous matrix with incorporated cells may be further cultured under suitable conditions, in a suitable culture medium and for an appropriate amount of time to expand cell numbers to a desired level.

In certain embodiments, the method further comprises incubating the cells incorporated in the porous matrix for 1 day or greater, 2 days or greater, 3 days or greater, 7 days or greater, 14 days or greater, 21 days or greater or 28 days or greater. Other time periods are contemplated.

In certain embodiments the method comprises use of a bioreactor to form the gel in situ and/or to incubate the cells in the matrix for a period of time. The term “bioreactor” as used herein refers to a system for culturing cells and tissue, and typically includes an incubator for providing suitable temperature and/or atmospheric conditions and one or more other components, such as components involved in fluid management. Typically, a bioreactor will provide the cell culture reagents to incubate and/or grow cells.

Certain embodiments of the present disclosure provide a method of incorporating cells in a porous matrix, the method comprising:
applying a gellable medium to the porous matrix; and
applying cells and a gelling agent to the porous matrix with applied gellable medium to form a gel comprising the cells, wherein the formation of the gel from the gellable medium comprises a time period of 10 minutes or less after contact of the gellable medium with the cells;
thereby incorporating the cells in the porous matrix.

Certain embodiments of the present disclosure provide a method of incorporating cells in a porous matrix, the method comprising:
applying plasma and/or a derivative thereof to the porous matrix; and
applying cells and thrombin to the porous matrix with applied plasma to form a gel comprising the cells, wherein the formation of the gel comprises a time period of 10 minutes or less after contact of the plasma with the cells;
thereby incorporating the cells in the porous matrix.

In certain embodiments, a method as described herein is used to produce a product for producing skin, to produce a cultured skin product, to deliver cells to a subject, or to deliver one or more cell derived factors (eg a growth factor) to a subject. Other uses are contemplated.

Certain embodiments of the present disclosure provide a porous matrix comprising incorporated cells produced according to a method as described herein.

Certain embodiments of the present disclosure provide a porous matrix comprising a gel comprising cells, the gel comprising the cells being formed in situ from a gellable medium, wherein the formation of the gel from the gellable medium comprises a time period of 10 minutes or less after contact of the gellable medium with the cells.

Examples of porous matrices are as described herein.

In certain embodiments, the formation of the gel comprises a time period of 9 minutes or less, 8 minutes or less, 7 minutes or less, 6 minutes or less, 5 minutes or less, 4 minutes or less, 3 minutes or less, 2 minutes or less, or 1 minute or less after contact of the gellable medium with the cells. Other time periods are contemplated.

In certain embodiments, the formation of the gel comprises a time period for formation of the gel after contact of the gellable medium with the cells of 10 to 300 seconds, 20 to 300 seconds, 25 to 300 seconds, 30 to 300 seconds, 45 to 300 seconds, 50 to 300 seconds, 60 to 300 seconds, 90 to 300 seconds, 120 to 300 seconds, 150 to 300 seconds, 180 to 300 seconds, 210 to 300 seconds, 240 to 300 seconds, 270 to 300 seconds, 10 to 270 seconds, 20 to 270 seconds, 25 to 270 seconds, 30 to 270 seconds, 45 to 270 seconds, 50 to 270 seconds, 60 to 270 seconds, 90 to 270 seconds, 120 to 270 seconds, 150 to 270 seconds, 180 to 270 seconds, 210 to 270 seconds, 240 to 270 seconds, 10 to 240 seconds, 20 to 240 seconds, 25 to 240 seconds, 30 to 240 seconds, 45 to 240 seconds, 50 to 240 seconds, 60 to 240 seconds, 90 to 240 seconds, 120 to 240 seconds, 150 to 240 seconds, 180 to 240 seconds, 210 to 240 seconds, 10 to 210 seconds, 20 to 210 seconds, 25 to 210 seconds, 30 to 210 seconds, 45 to 210 seconds, 50 to 210 seconds, 60 to 210 seconds, 90 to 210 seconds, 120 to 210 seconds, 150 to 210 seconds, 180 to 210 seconds, 10 to 180 seconds, 20 to 180 seconds, 25 to 180 seconds, 30 to 180 seconds, 45 to 180 seconds, 50 to 180 seconds, 60 to 180 seconds, 90 to 180 seconds, 120 to 180 seconds, 150 to 180 seconds, 10 to 150 seconds, 20 to 150 seconds, 25 to 150 seconds, 30 to 150 seconds, 45 to 150 seconds, 50 to 150 seconds, 60 to 150 seconds, 90 to 150 seconds, 120 to 150 seconds, 10 to 120 seconds, 20 to 120 seconds, 25 to 120 seconds, 30 to 120 seconds, 45 to 120 seconds, 50 to 120 seconds, 60 to 120 seconds, 90 to 120 seconds, 10 to 90 seconds, 20 to 90 seconds, 25 to 90 seconds, 30 to 90 seconds, 45 to 90 seconds, 50 to 90 seconds, 60 to 90 seconds, 10 to 60 seconds, 20 to 60 seconds, 25 to 60 seconds, 30 to 60 seconds, 45 to 60 seconds, 50 to 60 seconds, 10 to 50 seconds, 20 to 50 seconds, 25 to 50 seconds, 30 to 50 seconds, 45 to 50 seconds, 10 to 45 seconds, 20 to 45 seconds, 25 to 50 seconds, 30 to 45 seconds, 10 to 30 seconds, 20 to 30 seconds, 25 to 30 seconds, 10 to 25 seconds, 20 to 25 seconds, 10 to 20 seconds, or a time period about one of the aforementioned time periods. Other time periods are contemplated.

In certain embodiments, the formation of the gel comprises a time period of 10 to 50 seconds after contact of the gellable medium with the cells. In certain embodiments, the formation of the gel comprises a time period of 25 to 50 seconds after contact of the gellable medium with the cells.

In certain embodiments, the formation of the gel comprises a time period of 10 to 300 seconds after contact of the gellable medium with the cells.

In certain embodiments, the formation of the gel comprises a time period of 10 to 300 seconds after contact of the gellable medium with the cells. In certain embodiments, the formation of the gel comprises a time period of one of 10 to 50, 20 to 50, 25 to 50, 30 to 50, 10 to 30, or 20 to 30 seconds after contact of the gellable medium with the cells.

In certain embodiments, the formation of the gel comprises a time period of 25 to 50 seconds after contact of the gellable medium with the cells. In certain embodiments, the formation of the gel comprises a time period of about 25 to 50 seconds after contact of the gellable medium with the cells.

Examples of cells are as described herein.

In certain embodiments, the cells comprise skin cells. In certain embodiments, the cells one or more of fibroblast cells and/or a precursor thereof, keratinocyte cells and/or a precursor thereof, hair follicle cells and/or a precursor thereof, sweat gland cells and/or a precursor thereof, sebaceous gland cells and/or a precursor thereof, melanocyte cells and/or a precursor thereof.

In certain embodiments, the cells comprise cells that produce a paracrine factor, such as a growth factor.

In certain embodiments, the cells comprise cells for forming part of a tissue or organ, such as a skin. In certain embodiments, the cells comprise cells for assisting with forming a structural component.

In certain embodiments, the cells comprise stem cells or a progenitor of a desired cell type.

In certain embodiments, the cells for incorporation comprise autologous cells derived from a subject.

In certain embodiments, the porous matrix comprises an average pore size of 100 μm or greater, 200 μm or greater, 300 μm or greater, 400 μm or greater, 500 μm or greater, 600 μm or greater, 700 μm or greater, 800 μm or greater, 900 μm or greater, or 1000 μm or greater. Methods for determining pore size are known in the art. In certain embodiments, the pore size comprises the size of the largest cross sectional diameter of a pore. Other pore sizes are contemplated.

In certain embodiments, the porous matrix comprises an average pore size in the range from 100 μm to 1 mm, 200 μm to 1 mm, 300 μm to 1 mm, 400 μm to 1 mm, 500 μm to 1 mm, 600 μm to 1 mm, 700 μm to 1 mm, 800 μm to 1 mm, 900 μm to 1 mm, 100 μm to 900 μm, 200 μm to 900 μm, 300 μm to 900 μm, 400 μm to 900 μm, 500 μm to 900 μm, 600 μm to 900 μm, 700 μm to 900 μm, 800 μm to 900 μm, 100 μm to 800 μm, 200 μm to 800 μm, 300 μm to 800 μm, 400 μm to 800 μm, 500 μm to 800 μm, 600 μm to 800 μm, 700 μm to 800 μm, 100 μm to 700 μm, 200 μm to 700 μm, 300 μm to 700 μm, 400 μm to 700 μm, 500 μm to 700 μm, 600 μm to 700 μm, 100 μm to 600 μm, 200 μm to 600 μm, 300 μm to 600 μm, 400 μm to 600 μm, 500 μm to 600 μm, 100 μm to 500 μm, 200 μm to 500 μm, 300 μm to 500 μm, 400 μm to 500 μm, 100 μm to 400 μm, 200 μm to 400 μm, 300 μm to 400 μm, 100 μm to 300 μm, 200 μm to 300 μm, or 100 μm to 200 μm.

In certain embodiments, the porous matrix comprises an average pore size of greater than 100 μm to 1000 μm. In certain embodiments, the porous matrix comprises an average pore size of greater than 200 μm to 500 μm.

In certain embodiments, the porous matrix is biodegradable in vivo. In certain embodiments, the porous matrix comprises a biodegradable polymer. In certain embodiments, the biodegradable polymer has a half life in vivo (for example as measure by the mass retained) of 30 to 120 days.

In certain embodiments, the porous matrix is non-biodegradable in vivo. In certain embodiments, the porous matrix comprises a non-biodegradable polymer.

In certain embodiments, the porous matrix includes a fibrous matrix.

In certain embodiments, the porous matrix comprises a fibrous matrix, a sheet, a mesh, a mat, a woven matrix, a cloth, a foam and/or a sponge.

In certain embodiment, the porous scaffold comprises a synthetic matrix. In certain embodiments, the porous scaffold comprises a natural matrix or a matrix derived from a natural product.

In certain embodiments, the porous scaffold is sterilisable. In certain embodiments, the porous scaffold is shelf stable.

In certain embodiments, the porous matrix comprises a fibrous porous matrix.

In certain embodiments, the fibrous porous matrix comprises fibres with a diameter in the range from 50 μm to 200 μm. Other sizes are contemplated.

In certain embodiments, the fibrous porous matrix comprises fibres with a diameter in the range from 60 μm to 100 μm.

In certain embodiments, the porous matrix is in the form of a sheet or a mat.

In certain embodiments, the sheet comprises a thickness of 1 mm or greater, 2 mm or greater or 3 mm or greater. In certain embodiments, the sheet comprises a thickness of at least 1 mm, at least 2 mm, or at least 3 mm. Other sizes are contemplated.

In certain embodiments, the sheet comprises a thickness in the range from 1 mm to 3 mm.

In certain embodiments, the cells applied to the porous matrix comprise 1×10⁴ or greater cells per cm² of the porous matrix. In certain embodiments, the cells applied to the porous matrix comprise 4×10⁴ or greater cells per cm² of the porous matrix. In certain embodiments, the cells applied to the porous matrix comprise 1×10⁵ or greater cells per cm² of the porous matrix. Other cell densities are contemplated.

In certain embodiments, the cells applied to the porous matrix comprise 1×10³ to 1×10⁶ cells per cm² of the porous matrix. In certain embodiments, the cells applied to the porous matrix comprise 1×10⁴ to 1×10⁵ cells per cm² of the porous matrix.

In certain embodiments, the porous matrix comprises one or more of a polyurethane, a collagen, a glycosaminoglycan, a collagen-GAG co-polymer and/or mixture, a mucopolysaccharide, a poly(alpha-hydroxy acid), a poly(lactic acid) and/or a structural isomer thereof, a polyglycolic acid and/or a structural isomer thereof, a polylactic-polyglycolic polymer, a chitosan, and a polycaprolactone. Other materials are contemplated.

In certain embodiments, the porous matrix comprises a biodegradable polyurethane.

In certain embodiments, the gellable medium forms a gel upon exposure of the gellable medium to a condition, treatment and/or agent. Examples of conditions, treatments and agents are as described herein.

Examples of gellable media include plasma, and/or a gellable derivative thereof, a fibrinogen, a polysaccharide (such as an alginate, an agarose, a chitosan, a pectate), a polymer (such as a polyvinyl alcohol polymer), and a protein (such as gelatine, collagen).

In certain embodiments, the gellable medium comprises an autologous gellable medium.

In certain embodiments, the gellable medium comprises plasma and/or a gellable derivative thereof. In certain embodiments, the derivative of plasma comprises a component, extract, or fraction of plasma. In certain embodiments, the derivative of plasma comprises a component comprising fibrinogen.

In certain embodiments, the cells and the gellable medium are applied to the porous matrix at the same time. For example, cells for incorporation may be resuspended in the gellable medium, such as plasma.

In certain embodiments, the gellable medium is applied to the porous matrix and subsequently the cells are applied to the porous matrix. For example, plasma may be applied to the porous matrix and a solution containing cells for incorporation added to the plasma applied to the porous matrix.

In certain embodiments, the gel is formed by using a gelling agent to form a gel from the gellable medium.

In certain embodiments, the gellable medium comprises plasma and/or a derivative thereof and the gelling agent comprises a fibrinogen-clotting serine protease, such as a thrombin. In certain embodiments, the gelling agent comprises a thrombin.

In certain embodiments, the gelling agent comprises an autologous gelling agent. In certain embodiments, the gelling agent comprises an autologous thrombin.

In certain embodiments, the porous matrix comprises one or more rounds of gel formation incorporating cells in the porous matrix. For example, a first layer of cells may be incorporated in the porous matrix, and after the gel has set (and optionally cultured to expand the cells), a layer of further cells may be incorporated in the porous matrix by formation of a gel in situ carrying the further cells.

In certain embodiments, the use of the thrombin comprises a plasma/thrombin ratio of 60:40 to 85:15. In certain embodiments, the use of the thrombin comprises a plasma/thrombin ratio of 75:25 to 85:15. In certain embodiments, the use of the thrombin comprises a plasma/thrombin ratio of 60:40 to 82:18. In certain embodiments, the use of the thrombin comprises a plasma/thrombin ratio of 75:25 to 82:18.

In certain embodiments, the thrombin comprises a concentration of 2-16 U per ml of plasma. In certain embodiments, the thrombin comprises a concentration of 2-8 U per ml of plasma. In certain embodiments, the thrombin comprises a concentration of 4-16 U per ml of plasma. In certain embodiments, the thrombin comprises a concentration of 4-8 U per ml of plasma. Other concentrations are contemplated. One unit of thrombin is typically defined as the amount of enzyme needed to cleave 1 mg of fusion protein in 16 hours to 95% completion at 20° C. in a buffer containing 25 mM Tris-HCl, pH 8.4, 150 mM NaCl, and 2.5 mM CaCl₂.

In certain embodiments, the porous matrix comprises keratinocyte cells which have been applied subsequent to the applying of fibroblast cells.

In certain embodiments, the cells in the gel are substantially uniformly distributed in the gel.

Certain embodiments of the present disclosure provide a porous matrix comprising a gel comprising cells, the cells in the gel being substantially uniformly distributed in the gel.

In certain embodiments, the porous matrix is used for delivering cells to a subject.

Certain embodiments of the present disclosure provide a product for delivering cells to a subject, the product comprising a porous matrix as described herein. Methods for delivering cells to a subject by way of an implantable matrix are known in the art, for example as described in Steinwachs et al (2012) Cartilage 3(1): 5-12. Methods for implantation are known in the art.

Certain embodiments of the present disclosure provide a method of treating a subject using a porous matrix or product as described herein.

In certain embodiments, the porous matrix or the product are used for treating a wound in a subject. Examples of wounds are as described herein.

Certain embodiments of the present disclosure provide a method of producing a product for forming skin.

Certain embodiments of the present disclosure provide a method of producing a product for forming skin, the method comprising:
incorporating fibroblast cells and/or keratinocyte cells, and/or progenitors thereof, in a porous matrix by forming one or more gels comprising the cells in situ in the porous matrix from a gellable medium, wherein the formation of the one or more gels from the gellable medium comprises a time period of 10 minutes or less after contact of the gellable medium with the cells; and
incubating the porous matrix with incorporated fibroblast cells and/or keratinocyte cells, and/or progenitors thereof, to expand numbers of cells;
thereby producing a product for forming skin.

Certain embodiments of the present disclosure provide a method of producing a product for forming skin, the method comprising:
applying plasma to a biodegradable porous matrix;
applying fibroblast cells, and/or a progenitor thereof, and thrombin to the biodegradable porous matrix with applied plasma to form a gel comprising fibroblast cells incorporated in the porous matrix;
incubating the porous matrix with incorporated fibroblast cells;
applying keratinocyte cells, and/or a progenitor thereof, and thrombin to the porous matrix with incorporated fibroblast cells to form a gel comprising keratinocyte cells incorporated in the porous matrix; and
incubating the porous matrix with incorporated fibroblast cells and keratinocyte cells;
wherein the formation of the said gels comprises a time period of 10 minutes or less after contact of the plasma with the cells;
thereby producing a product for forming skin.

Certain embodiments of the present disclosure provide a product for forming skin produced according to a method as described herein.

Certain embodiments of the present disclosure provide a cultured skin product produced according a method as described herein.

Certain embodiments of the present disclosure provide a method of forming skin in/on a subject, the method using a product as described herein to form skin in/on the subject.

In certain embodiments, the method of forming a skin in/on a subject comprises using autologous cells.

Certain embodiments of the present disclosure provide a method of treating a wound in a subject, the method comprising applying a product for forming skin as described herein to the wound in the subject.

In certain embodiments, the wound comprises a burn, a post-operative wound, a chronic wound, or a diabetic ulcer. Examples of wounds are as described herein.

Certain embodiments of the present disclosure provide a method of producing a product for forming skin in a subject, the method comprising:
applying autologous plasma, and/or a derivative thereof, to a biodegradable porous matrix;
applying autologous fibroblast cells, and/or a progenitor thereof, and thrombin to the biodegradable porous matrix with applied plasma to form a gel comprising fibroblast cells incorporated in the porous matrix;
incubating the porous matrix with incorporated fibroblast cells;
applying autologous keratinocyte cells, and/or a progenitor thereof, and thrombin to the porous matrix with incorporated fibroblast cells to form a gel comprising keratinocyte cells incorporated in the porous matrix; and
incubating the porous matrix with incorporated fibroblast cells and keratinocyte cells;
wherein the formation of the said gels comprises a time period of 10 minutes or less after contact of the plasma with the cells;
thereby producing a product for forming skin in a subject.

Certain embodiments of the present disclosure provide a method of treating a wound in a subject, using a porous matrix or a product as described herein.

Examples of wounds are as described herein.

In certain embodiments, the method of treating a wound comprises applying a seal to the wound to close the wound and/or reduce evaporative water from the wound.

For example, a polyurethane membrane (with or without a porous matrix) can be used to seal a wound prior to application of the cultured skin product.

Methods for applying a seal and/or applying a cultured skin product are known in the art and described herein.

Certain embodiments of the present disclosure provide a method of treating a wound in a subject, the method comprising:
applying a seal to the wound to close the wound and/or reduce evaporative water from the wound;
removing the seal from the wound; and
applying a product as described herein to the wound;
thereby treating the wound.

Certain embodiments of the present disclosure provide a method of expanding autologous cells for introduction into a subject.

A porous matrix or product as described herein may be used to provide a carrier so as to be able to expand cells numbers to a desired level for introduction into a subject.

Certain embodiments of the present disclosure provide a method of expanding cells for introduction into a subject, the method comprising:
incorporating cells in a porous matrix by forming a gel comprising the cells in situ in the porous matrix from a gellable medium, wherein the formation of the gel from the gellable medium comprises a time period of 10 minutes or less after contact of the gellable medium with the cells; and
incubating the porous matrix to expand the cells.

In certain embodiments, the cells are autologous cells.

Certain embodiments of the present disclosure provide a method of expanding autologous cells for introduction into a subject, the method comprising:
incorporating autologous cells in a porous matrix by forming a gel comprising the cells in situ in the porous matrix from a gellable medium, wherein the formation of the gel from the gellable medium comprises a time period of 10 minutes or less after contact of the gellable medium with the cells; and
incubating the porous matrix to expand the cells.

Certain embodiments of the present disclosure provide cells expanded by a method as described herein. In certain embodiments, the cells comprise skin cells.

Certain embodiments of the present disclosure provide a system for producing cultured skin.

Certain embodiments of the present disclosure provide a system for producing cultured skin, the system comprising:
(i) a product comprising a porous matrix with incorporated cells, the cells being incorporated in the porous matrix by forming a gel comprising the cells in situ in the porous matrix from a gellable medium, wherein the formation of the gel from the gellable medium comprises a time period of 10 minutes or less after contact of the gellable medium with the cells; and
(ii) a bioreactor for culturing the product to produce cultured skin.

In certain embodiments, the product comprises a sheet of the porous matrix with incorporated cells.

In certain embodiments, the sheet has a size of greater than 400 cm².

Certain embodiments of the present disclosure provide a method of producing cultured skin using a system as described herein.

Certain embodiments of the present disclosure provide a composition comprising a porous matrix with incorporated cells, as described herein.

In certain embodiments, the composition is a therapeutic composition.

In certain embodiments, the composition is used to deliver cells to a subject. In certain embodiments, the composition is used to deliver one or more cell derived factors to a subject.

Porous matrices are as described herein.

For example, a composition may comprise a porous matrix with incorporated cells in the form of beads and/or particles.

Certain embodiments of the present disclosure provide a kit for performing a method as described herein.

In certain embodiments, the kit is used for producing a cultured skin product.

The kit may include one or more reagents as described herein, and/or instructions for performing a method as described herein.

For example, a kit may comprise the porous matrix, and one or more of enzymes, buffers, serum, additives, stabilisers, diluents, culture media, agents, growth factors, consumables, tissue culture vessels, plates, flasks, and instructions.

Certain exemplary embodiments are illustrated by some of the following examples. It is to be understood that the following description is for the purpose of describing particular embodiments only and is not intended to be limiting with respect to the above description.

### EXAMPLE 1

### Materials

(i) Animals

Three large white/landrace cross domestic pigs (Sus scrofa) initially weighing 22.2-33 kg were acclimatised for one week prior to study commencement. Housing and animal care were provided in accordance with NHMRC Guidelines.

(ii) Biodegradable Polyurethane Matrices

Matrices were provided by PolyNovo Biomaterials Pty. Ltd (Port Melbourne, Victoria, Australia) as 10×10 cm pieces, sterilized by gamma-irradiation and dry-packed.

The scaffold for the production of the composite cultured skin was a 1 mm thick, unsealed foam matrix 10×10 cm in size.

### EXAMPLE 2

### Isolation and Culture of Cells for Composite Cultured Skin (CCS) Creation

Split thickness skin biopsies were harvested from each pig. Skin samples (˜8×8 cm) were washed thoroughly in multiple povidone-iodine, gentamicin and phosphate buffer saline solutions, cut into small pieces and incubated overnight in Dispase II (6 mg/ml) at 4° C. The epidermis was separated and processed for keratinocytes.

Basal keratinocytes were isolated by trypsinising the epidermal sheets with 0.05% Trypsin-EDTA (Sigma; St Louis, Mo.) with gentle agitation for 5 minutes. This solution was then quenched with equal volumes of Soybean trypsin inhibitor (Sigma; St Louis, Mo.). Keratinocytes were co-cultured with irradiated Swiss albino (iSA3T3) fibroblasts in keratinocyte growth media, SEL-KGM-1% FBS (based on Rheinwald and Green's original media). The dermal component of the biopsy was required to isolate the fibroblasts. It was cut into smaller (0.3 cm²) pieces and further digested with Collagenase I (3 mg/ml) at 37° C. with gentle agitation until the dermal pieces were virtually digested (3-5 hours depending on thickness of dermis). After several washes, the cells were centrifuged and cell counts performed. Fibroblasts were cultured in DMEM-10% FBS medium supplemented with antibiotic/antimycotic (Sigma; St Louis, Mo.). Cells were incubated at 37° C. with 5% CO₂ and the medium changed every 2-3 days.

### EXAMPLE 3

### Thrombin Isolation from Plasma

(a) Reagents
25 ml plasma
1 L bag sterile water for injection
0.25% acetic acid
100 ml bag saline
8.4% sodium bicarbonate buffer
10% calcium chloride
(b) Thrombin isolation from Plasma
Thaw 25 mls plasma in waterbath (37° C. and no longer than 30 mins)
Prepare diluted acetic acid solution
Using a plasma transfer set add 175 mls of sterile water (1 L bag sterile water for injection bag) to a 250 ml centrifuge tube.
Add 35 mls of 0.25% acetic acid to the tube using a 25 ml pipette.
Add 25 mls of plasma to the tube to dilute acetic acid
Mix well and allow to sit at least 10 seconds for precipitation to occur
Centrifuge at 3200 rpm for 5 mins using a low brake, set at 2.
Prepare bicarbonate buffer
Spike a 100 ml bag of isotonic saline with a transfer set.
Using a 1 ml syringe and 23 g needle draw up 0.5 ml of 8.4% sodium bicarbonate and inject into bag.
Use a 10 ml syringe and draw up 5 ml of 10% calcium chloride and mix contents.
Carefully decant the acetic acid supernatant form the step above, into waste containers, remove last drops of acetic acid with sterile syringe and cannula leaving behind the pellet.
Draw up 7-10 mls from the thrombin bicarb buffer bag and add to the 250 ml centrifuge tube pellet to dissolve.
Using a cannula and syringe transfer the solution to a red top blood collection tube, mix and transfer to a second red-top tube, mix and transfer to a 15 ml centrifuge tube.
Incubate at 37° C. (lay tube on side to identify when coagulation starts), record time on worksheet.
Check after 30 mins, if a coagulum is observed perform check, if no coagulum then incubate further 15-30 mins regularly checking.

### EXAMPLE 4

### Gel Formation Times and Effect on Keratinocyte Growth

Initial experiments with plasma and thrombin were used to determine gel setting times as measured in 6 well culture plates. The data is shown in the Table below:

### Plasma Thrombin Ratio

| | 75:25 | 60:40 | 50:50 |
|---|---|---|---|
| Gel Setting Time | ~50 sec-1 min | ~30 sec | ~20 sec |

The effect of gel setting time and plasma/thrombin ratio on cell growth in the gel was determined in 6 well culture plates. Gels were formed using the selected ratio of plasma to thrombin and 2.5×10⁴ cells. In this study, keratinocytes were used as these cells provided a more sensitive measure of cell growth than fibroblasts.

### Plasma Thrombin Ratio

| | 75:25 | 60:40 | 50:50 |
|---|---|---|---|
| Gel Setting | ~50 sec-1 min | ~25 sec-30 sec | ~20 sec |
| Time | | | |
| (Day 0) | | | |
| Day 2 | Scattered growth | No outgrowth of | No outgrowth of |
| | of cells, | cells, no | cells, majority |
| | cells attaching | attachment | of cells |
| | and starting to | to bottom of | rounded |
| | proliferate | well | |
| Day 4 | Cell colonies | Some growth of | Limited cell |
| | present and in | cells | growth, no |
| | reasonable number | | attachment |
| Day 7 | Continued cell | some growth of | Limited growth, |
| | growth on bottom | cells | no attachment |
| | of well | | |

The studies confirmed that a gel formation time of 2 minutes or less was likely to provide an advantageous time for use with cells. These studies also confirmed that a gel formation time of 25 seconds to 1 minute/30 seconds to 1 minute/25 seconds to 50 seconds appeared to provide gel characteristics that were most favourable for cell proliferation and outgrowth.

### EXAMPLE 5

### Use of Commercial Thrombin

A source of commercial thrombin (ProSpec) was obtained and clotting times tested (cat #PRO-617, lot #313PPTHROM).

Testing of clotting times for pig thrombin
500 U diluted with 1 ml of sterile water and 0.1% HSA (carrier protein) and stored according to manufactures instructions. Stored in 40 μl aliquots and frozen. Therefore 500 Units/ml.
Testing clotting times of diluted thrombin
For further testing thrombin was diluted 1/10 with sterile H₂0 and then used accordingly.

| Thrombin | | Thrombin | Plasma | | Thrombin | | |
|---|---|---|---|---|---|---|---|
| volume | | Units | volume | | U/ml plasma | Time to clot | |
| 20 | μl | 1 | 500 | μl | 2 | >1 | minute |
| 4 | μl | 0.2 | 50 | μl | 4 | 30-40 | secs |
| 4 | μl | 0.2 | 25 | μl | 8 | 25-30 | secs |
| 4 | μl | 0.2 | 12.5 | μl | 16 | 15-20 | secs |

Testing of Thrombin dilutions with Foam and Fibroblasts
Thrombin-Plasma concentrations tested

Thrombin μl/plasma μl
0.08 μl/μl
0.16 μl/μl
0.32 μl/μl

Pig Fibroblasts used at 6×10⁴/cm²
   2× T175's harvested at 60% confluence
   Cell count (4 squares in 10 mls) 52(2)=54, 96.3% 26×10⁴/ml.
Pig Plasma used
6 well plates-duplicates
Tested 3 different polyurethane matrices (1×1 cm²)
   #1 TM8-93 (1 mm TM8-93, Square 10×10 cm² all individually packed sterile)
   #2 TM8-93 (1.5 mm TM8-93, square with rounded corners, ˜13×12 cm², in 1 pack sterile)
   #3 PN005C-407 (1 mm foam sheet square 10×10 cm²)
200 μl of plasma used per piece
4 plates set up
   1. 16 μl/200 μl (7.4:92.6)
   2. 32 μl/200 μl (13.8:86.2)
   3. 64 μl/200 μl (24:76)
   4. PLASMA ONLY
Foams soaked in 200 μl of plasma set up for 10 mins
Let soak for 30 minutes
Foams flipped and left another 30 mins
Then transferred into new plates. Note: not all 200 μl plasma absorbed the left over/extra plasma added on top by drop. 1—˜50 μl, 2—25 μl, 3—75 μl. Then left for another 30 minutes.
Excess from 1 and 3 removed ˜25 μl and 50 μl respectively.
Duplicate wells were set up for each condition and the total thrombin volume was divided by 9 and then thrombin and cells added evenly with a multipipette (Plate 1—1.77 μl×9=16 μl. Plate 2—3.55 μl×9=32 μl. Plate 3—7.11 μl×9=64 μl).
Plates then left for 30 mins to set in hood and transferred to incubator for 10 minutes before adding 2 mls of media.

Results
Foam #2 soaked up plasma best as it felt a bit thicker
Excess plasma from 1 and 3 removed from edges of foam, ˜25 μl and 50 μl respectively.
   0.08 μl/μl Actual 16 μl/175 μl (8.4:91.6)
   0.16 μl/μl Actual 32 μl/200 μl (13.8:86.2)
   0.32 μl/μl Actual 64 μl/150 μl (30:70)
Live/dead staining of fibroblasts in foams observed at Day 7

| DAY 7 | | Foam #1 | Foam #2 | Foam #3 |
|---|---|---|---|---|
| PLATE 1 - | Light and | Some loss of cells, | Minimal cells near | Some cell loss, |
| 0.08 μl/μl | confocal | majority on bottom | bottom of foam. | most cells noted |
| Clotting | Microscopy | of well and near | Cells present closer | near bottom of foam |
| Time (CT) | Observations | bottom of foam. | to top in PG clump. | with majority on the |
| 30-40 secs | | Not many cells | Possibly due to | bottom of well in a |
| | | present. | foam thickness. | PG layer. |
| PLATE 2 - | Light and | Some cells on | Minimal cells near | Good growth. Cells |
| 0.16 μl/μl | confocal | bottom of well but | bottom of foam due | in bottom area of |
| CT 25- | Microscopy | also a reasonable | to the increase in | foam ~90 μl in. |
| 30 secs | Observations | amount seen in | thickness, also some | When flipped good |
| | | ‘middle’ to bottom | debris on foam | cell layer and cell |
| | | of foam and | cutting edges. When | numbers ~80 μl |
| | | sections with a lot | foam flipped for | down, looks like |
| | | of cells. Minimal | viewing most cells | this all over. More |
| | | cells at bottom of | noted on top of | cells than near |
| | | foam. | foam ~80-100 μl in | bottom of foam, |
| | | | from top of foam. | most pores filled |
| | | | | ~75 μl in. |
| PLATE 3 - | Light and | Minimal cells on | Minimal growth | Cells limited to |
| 0.32 μl/μl | confocal | outside of well, not | outside wells. Cells | periphery of foam, |
| CT 15- | Microscopy | as many as plate 1 | mainly near top of | good containment |
| 20 secs | Observations | and 2. Cells seen | foam, but maybe | and good growth |
| | | within foam | rounded. When | within foam. Cells |
| | | ~middle limited to | flipped most cells | in middle to top but |
| | | bottom and hard to | close to top of foam | not evenly |
| | | tell at top appears to | cells at ~80-100 μl | distributed. When |
| | | be good growth. | in from top | flipped a lot more |
| | | When flipped | reasonably even and | cells present at top |
| | | showed pretty even | distributed over that | of foam ~80 μl |
| | | growth consistency | level. | in/down, even |
| | | over all foam. | | observed at 30-60 μl. |

Conclusions

Plate 3 (15-20 secs CT): Showed minimal to no cell growth at bottom of foam mainly plasma gel observed. A reasonable number of cells noted at top for all three different foams. Foam #3 appears to be the choice of foam as #2 shows signs of some cytotoxicity to the cells as they presented rounded in areas and a lot of debris could be seen on the edges of the foam pores which may be deleterious to cell attachment and growth.

Plate 2 (25-30 secs CT): Show decrease cell numbers near the bottom of the foam and on the well plate, indicating the majority had been retained in the upper sections of the foam.

Plate 1 (30-40 secs CT): Showed majority of cells were near the bottom of the foam or on the bottom of the well plate for plate 1 independent of foam type.

For the application of incorporating skin cells in a porous matrix, a setting time between 15-30 secs and Foam #3 was found to yield the best cell growth and cell distribution throughout the foam.

### EXAMPLE 6

### A Gel System for Incorporating Cells into a Porous Matrix

A plasma gel system was developed to incorporate cells into the scaffold. This involved the use of one bag (265 ml) of blood, aseptically collected from the femoral artery of each pig, for autologous plasma and thrombin isolation. Aliquots of the plasma and thrombin were frozen for composite manufacture.

A 10×10 cm, 1 mm thick and unsealed foam matrix was used, being the basis of a cultured composite skin. In a sterile dish, the matrices were initially pre-soaked in 5 ml plasma. Complete wetting of all foam surfaces and inner pores was undertaken.

Plasma soaked foam was placed into the culture dish. An additional 1 ml of plasma was added to fill any large pores evident within the foam.

(i) Addition of Fibroblasts—Stage 1

Fibroblast cell suspension was prepared (4×10⁴ cells per cm² of matrix). Fibroblasts were resuspended in 2 mls of thrombin using an 75:25—plasma:thrombin ratio.

The cell suspension was drawn up using a 3 ml syringe and drawing up needle and then slowly added drop wise onto the plasma-soaked matrix. A gel was subsequently formed within 20 to 30 seconds, and this was allowed to completely set in the hood for 10 minutes then the matrix was carefully moved to an incubator for another 20 minutes before media addition.

In a culture dish with foam, 500 mls of media was slowly (using a peristaltic pump system add media in at 5 mls per minute). Pump was set to 2 ml/minute and left until media change.

Media was change every 2-3 days and cultured for 5-7 days.

(ii) Addition of Keratinocytes—Stage 2

A keratinocyte cell suspension was prepared (2.5×10⁵ cells per cm² of matrix).

Keratinocytes were resuspended in 1.4-1.6 mls of thrombin.

The cell suspension was drawn-up using a syringe and drawing up needle and then slowly added drop wise. A gel was subsequently formed within 20-25 seconds and this was allowed to set in a hood for 10 minutes and then carefully moved to the incubator for another 20 minutes before media addition.

In a culture dish with the matrix foam was add 500 mls of media slowly (using the peristaltic pump system media was added in at 5 mls per minute). The pump was used at 2 ml/minute.

Media was changed every 2-3 days and cultured for another 9-12 days.

For transplantation, media was drained from the culture vessel and the CCS transported to the theatre for application.

One 10×10 cm composite was randomly assigned as a ‘spare’ for serial analysis during the culture period. At different time points, biopsies from this sample were stained with viability dyes (Calcein/Ethidium, Sigma; St Louis, Mo.) and analysed using a Nikon Confocal Microscope. Light micrographs were also obtained during media changes using an Olympus C-5060 digital camera and microscope lens adapter.

### EXAMPLE 7

### A Polyurethane Seal for Temporising Wounds

A gamma-irradiated sterilized biodegradable temporising matrix (BTM) was provided by PolyNovo Biomaterials Ltd (Port Melbourne, Victoria, Australia). The BTM is a 2 mm thick foam matrix with a 150 μm thick, non-biodegradable, microporous polyurethane ‘sealing’ membrane, bonded to its superficial surface.

### EXAMPLE 8

### Treatment of Wounds

Materials and Methods

(i) Porcine Wound Model

The surgical and dressing protocols for this study were performed as previously described in Greenwood and Dearman (2012) J Burn Care Res. 33:7-19.

Four 8×8 cm sites were designed on the flanks of the pig. Split thickness skin grafts ( 12/1000th of an inch) were taken from each animal to provide autologous components for keratinocyte cell culture. The donor site wounds were then deepened to the panniculus adiposus and 2 mm-thick, sealed BTM polymers were implanted affixed with surgical staples.

(ii) Treatment Allocations

The treatment sites in this study were as follows: 1) & (2) a sealed BTM with CCS applied after BTM integration at Day 28. (3) a sealed BTM with split thickness skin graft applied at Day 28. (4) a fresh wound (to panniculus adiposus) created at Day 28 with CCS applied

Two of the BTM treated sites received cultured composite skin at Day 28 and the other a split thickness skin graft taken from area four (initially untreated). This fourth (donor) site was then deepened to a full-thickness wound and grafted with a CCS only on Day 28.

(iii) CCS Application

The BTM treatment sites were delaminated of residual seal and dermabraded with a diamond burr to refresh the superficial surface of the wound, preparing them for composite application. The composites were carefully applied, trimmed to size and affixed with surgical steel staples. An additional Mepitel piece, pre-cut to each individual treatment area, was affixed rostrally with staples to minimise shear of the composites. The first dressing change allowed retention of this ‘under-dressing’. A large Mepitel (20×20 cm) dressing then covered all sites and standard procedures followed. Dressing changes occurred twice a week.

(iv) Wound Assessments

Wounds were cleaned, dressed and visually assessed for infection, matrix integration and re-epithelialisation. For macroscopic evaluation digital photographs were taken with a Canon EOS550D SLR digital camera. Length, girth and weight were measured to monitor pig condition. Wound areas were measured using the Visitrak™ system (Smith & Nephew Ltd, Hull, UK) and evaporative water loss was assessed using a Vapometer (Delfin Technologies Ltd., Helsinki, Finland). To minimise any disruption of the treatment site, tracings and readings were not obtained on day three after treatment.

(v) Histological Analysis

Punch biopsies were obtained for histological assessment and large, post-mortem, full-thickness excision biopsies were collected at necropsy on Day 42 post-surgery. These samples were fixed in neutral-buffered formalin. A number of staining methods [Hematoxylin and Eosin (H&E), Periodic acid Schiff (PAS), Immunohistochemical and fluorescence (BovK; 1:500, Dako; Z0622) for keratin] were employed to visualise and confirm the quality of re-epithelialisation, basement membrane formation, granulation tissue formation, fibroblast influx, in-growth of blood vessels and collagen deposition. The histology slides were reviewed by an independent pathologist.

Results

(i) BTM Before CCS Application

At initial surgery, sealed BTMs were applied to the three treatment areas with seal delamination scheduled for Day 28 post-application. However, in a number of sites seal delamination occurred before day 28, with partial seal removal and varying degrees of seal fragmentation with or without superficial granulation. Only two sites maintained a seal that could be delaminated in one whole piece on Day 28. Due to the sealing shortfalls, the wounds had contracted to 50% of their original size (mean 50.5%). Since proof of concept of CCS take over integrated BTM foam was the only outcome sought in this study, application of the cultured composite skins to the treatment areas were selective and the split thickness skin grafts, taken from the fourth site, were applied to the smallest residual integrated BTM wound.

BTM with STSG

Partial STSG take was observed in 1 of the 3 pigs with the remaining grafts completely successful (100% take). They appeared normal and healthy, vascularising early and maintaining wound size from application to endpoint. Histological analysis showed the engraftment of normal STSG over the integrated 2 mm BTM.

BTM with CCS

The cultured composite skins were easy to manipulate, apply, trim and affix with steel staples. They conformed to the wound area and were flush to the wound edge. The CCS was easily cut to size and no tearing was observed on their subsequent application with staples.

Proof of Concept

Successful ‘take’ of the cultured composite skin on an integrated 2 mm BTM wound. Random punch biopsies taken throughout the study indicated that epithelium was present from Day 3 post-application.

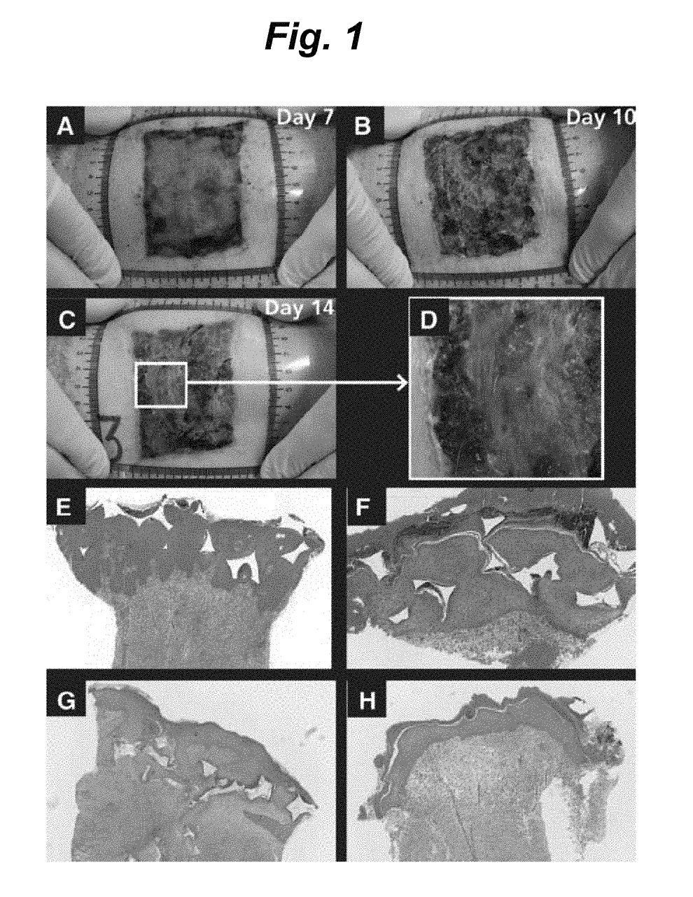

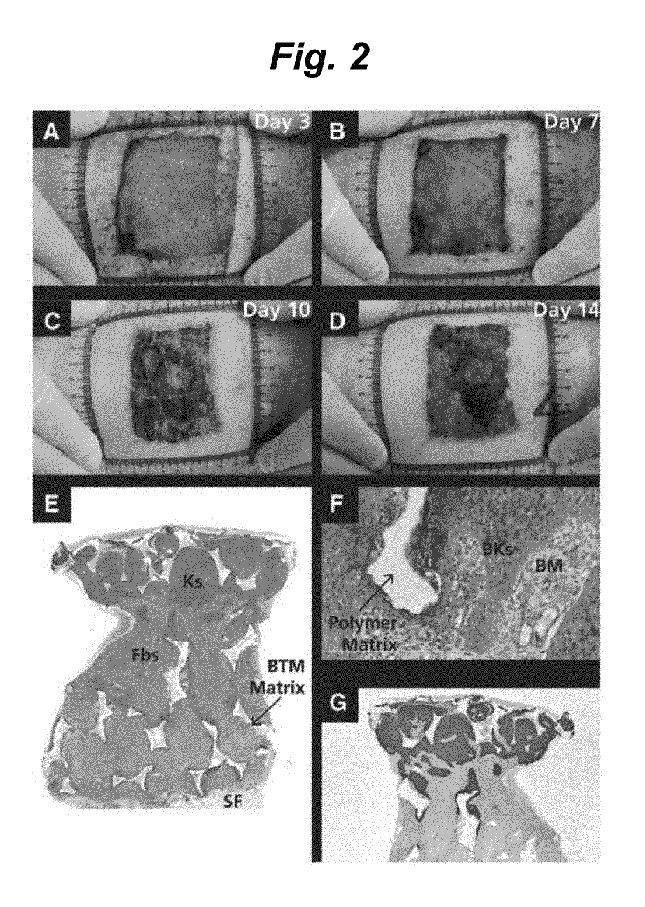

Of the CCS sites, clinically four did not ‘take’ and were removed on Day 7 post-application, however, upon removal there appeared to be a small amount of residual foam on the surface indicating that the deeper component of the CCS had adhered to the wound. The two sites that demonstrated the proof of concept with successful ‘take’ of the cultured composite skin on an integrated 2 mm BTM wound were from Pig 2 (FIGS. 1 and 2).

Clinical progression from day 3 post application to day 14 is shown in FIG. 1A-C and FIG. 2A-D, displaying CCS vascularisation and integration by Day 10. Clinically, these wounds displayed a desiccated appearance of the superficial surface polymer matrix, forming a stiff carapace over the underlying wound surface, which, when lifted, showed several areas of epithelium below (FIG. 1B-D).

Furthermore, histological analysis demonstrated a well-developed epithelium integrated around and within the foam at Day 7, 10 and 14 (FIG. 1E-G). The integration of the 2 mm BTM on subcutaneous fat with the superficial 1 mm CCS ‘take’ was evident upon histological analysis (FIG. 2E), however the degree of 2 mm BTM ‘take’ varied as discussed earlier. A defined basement membrane was present by Day 7 (FIG. 2F) and the presence of a thick keratinised layer above the epithelium was confirmed by staining with BovK (FIG. 2G). The degree of integration of the 1 mm CCS also varied. There were areas where no CCS was visible and the polymer had apparently ‘shed’ from the healed surface, after depositing cells, thus acting as a delivery vehicle (FIG. 1H). Partial CCS integration was exhibited in some wounds, with a deep layer of integrated CCS with surrounding epithelium and a superficial unintegrated matrix layer, or complete integration (‘take’), where the entire CCS was surrounded and incorporated within an epithelium.

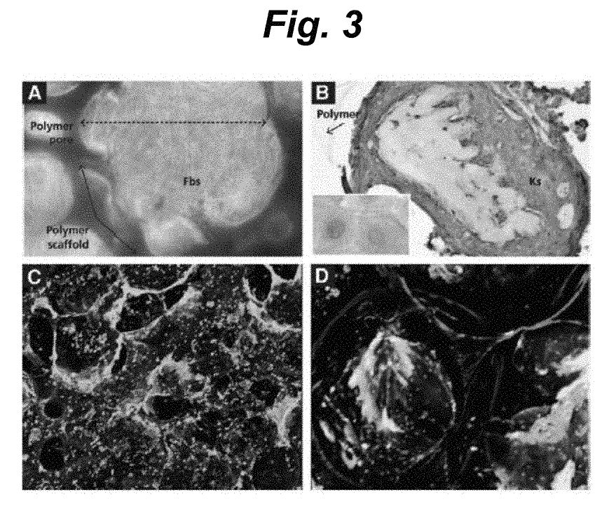

Light micrograph photos during culture and prior to application show a scattering of fibroblasts in and around the foam structure (FIG. 3A). Following keratinocyte application to the fibroblast plasma-gel matrix, light microscopy demonstrated the development of keratinocyte sheets in areas. This was confirmed by immunohistochemical staining for cytokeratin on the spare CCS (FIG. 3B). Immunofluorescence was performed and showed numerous keratinocytes (stained positive with BovK-green) and counter-stained fibroblast nuclei (with propidium iodide-red, FIG. 3C). The majority of cells had settled in the deeper section of the composite as revealed by confocal microscopy. Cell detachment from the foam edge, and clumping, was evident from the stained composite (FIG. 3D), possibly due to lifting and stretching of the CCS from the culture vessel prior to application. During histological analysis of the spare CCS, vertical cutting (from superficial to deep) through the foam proved difficult. In an attempt to confirm the presence of a stratified epithelium on the CCS, horizontal sectioning was performed (cuts parallel to the superficial surface).

Cultured Composite Skin Alone (no BTM)

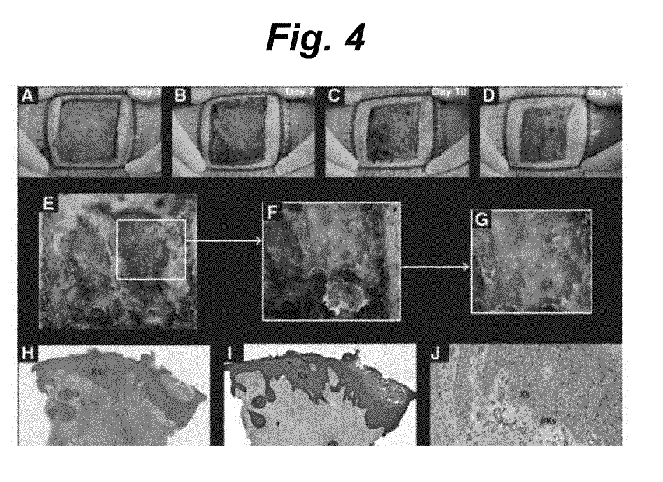

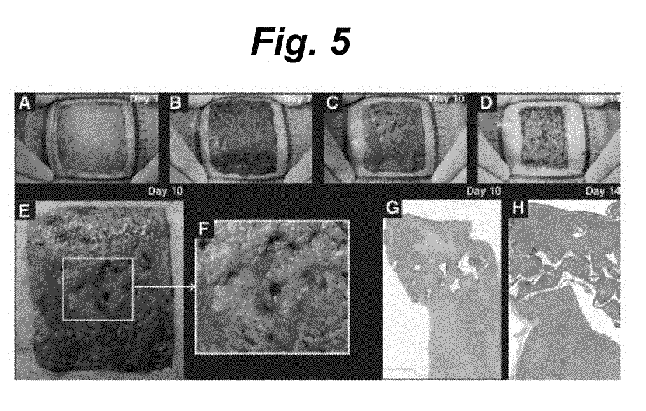

Applying a 1 mm thick composite cultured skin directly to a wound bed i.e. without any BTM revealed that the composite is capable of two different actions: 1. Acting as a delivery vehicle, with cells being deposited from the composite, which enabled cell attachment and development of an epithelium, whilst the foam itself was ‘shed’ from the wound surface (FIG. 4). Composite ‘take’, (an integrated skin replacement) 14 days post application (Day 42) with a neo-epithelium integrated within the foam (FIG. 5).

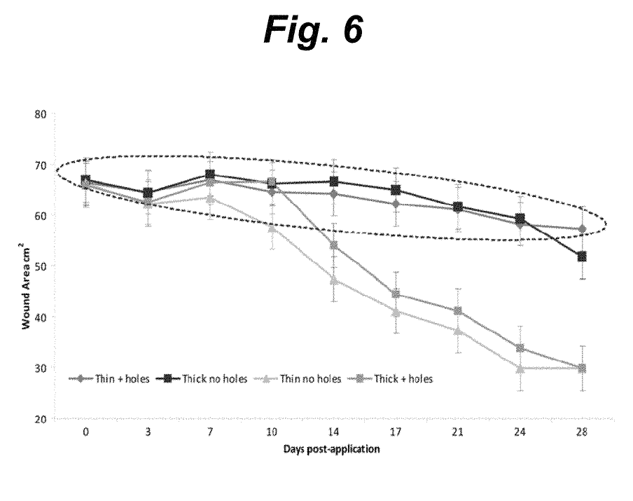

The composite applied directly to the wound bed for Pigs 1-3 illustrates these different outcomes. FIG. 4A-D demonstrates a temporal sequence following composite application into a fresh wound. The CCS alone from Pig 1 demonstrated a similar overlying ‘stiff carapace’ phenomenon, as seen in some of the BTM-CCS wounds. Clinically by Day 10, the majority of the wound (FIG. 4E) displayed a dark staining (due to silver deposits from the Acticoat dressing), that, when lifted, (FIG. 4F) displayed a matt, robust epithelium (FIG. 4G). On histological analysis, sections from Pig 1 had no polymer matrix visible, but a well-developed keratinised epidermis had formed, closing the wound (FIG. 4H,I). A well-defined basement membrane was demonstrable by Day 7 (FIG. 4J). This CCS had thus acted as a ‘delivery vehicle’ for cells. This was in contradistinction to Pig 2, which displayed composite skin ‘take’ (FIG. 5A-D). Clinically the cultured composite from Pig 2 completely integrated by Day 7 with no polymer structure visible and exuberant vascularity (FIG. 6B) and 80% clinically re-epithelialised by Day 10 (FIG. 5C). This wound was supple to handle and appeared almost completely healed (96%) with a flaking keratin surface by Day 14 post-application (FIG. 5D), again with some silver staining. No matrix ‘shedding’ was seen in this wound. Clinically, central re-epithelialisation was clearly evident upon examination at Day 10 (FIG. 5E,F) and confirmed with a punch biopsy displaying a well developed epithelium and incorporated lmm CCS (FIG. 5G). H&E sections from Day 14 also confirmed ‘take’ with epithelial integration around the matrix (FIG. 5H). Pig 3 similarly demonstrated foam integration, however re-epithelialisation was not as prominent as Pigs 1 and 2. Pig 3 histology showed plasma cell infiltration on the surface at Day 7. By Day 14, clinically, 70% of the wound had re-epithelialised. A further 20% was covered by a polymer crust, which exhibited underlying epithelium. The CCS here had clinically acted as a delivery vehicle for cells rather than demonstrating composite ‘take’.

At Day 14 post-application, the mean evaporative water readings for Pigs 1-3 was 29.9 g/m2 for the composites. Skin graft readings were comparable to the composites alone (no BTM) at Day 14 post-CCS application, with a mean reading of 20.4 g/m2, indicative of stratum corneum formation. The evaporative water loss for the BTM-CCS wounds showed a steep decline from Day 7 post application (241 g/m2 to 72.9 g/m2). Whilst the results suggest a trend of high evaporative water loss within the first week of composite application, with a subsequent fall in evaporative water loss in the following week, the small numbers involved preclude meaningful statistical analysis.

### EXAMPLE 9

### Optimisation of a Polyurethane Dermal Matrix and Experience with a Polymer-Based Cultured Composite Skin

Methods

(i) BTM Optimisation Studies

Groundwork studies investigating the BTM seal were required before the main study could commence. Ten variants of BTM seals consisted of wound generation and implantation, with repeated dressing changes followed by final seal delamination between days 21-28 and final wound evaluation. Fifteen pigs were utilised for the optimisation studies. A total of 44 wound sites received BTMs with varying seal properties. The polymer and BTM structure itself was not modified for any of these studies.

The BTM consisted of an integrating, biodegradable, polyurethane foam dermal matrix and a temporary, non-biodegradable polyurethane seal (or lamina). The seal minimises evaporative water loss and resists contraction during matrix integration. Delamination refers to the separation of the seal from the BTM. This process is usually planned by the surgeon when the dermal component has integrated fully and prepares the superficial surface of the neodermis for definitive closure. The seal variants ranged in thickness from 50-150 μm, some seals were perforated or non-perforated and a commercial seal was also tested (Table 1). The seal/matrix bonding methods were also evaluated.

TABLE 1
BTM seal optimization studies.

| | | | | | Statistical | | Mean Water |
|---|---|---|---|---|---|---|---|
| | | | Mean | Mean | Significance | | Loss Before |
| | | | Initial | Final | Between | | Seal |
| | | Wound | Wound | Wound | Initial and | Percentage | Delamination/ |
| | | Sites | Size | Size | Final Wound | of Wound | Removal |
| Study and Seal type | | (n) | (cm²) | (cm²) | Size | Contraction | (g/m²h)^{#} |
| Thickness of seals, μm | 50 | 3 | 63.8 | 45.9 | P < 0.0001 | 28 | 33 |
| | 100 | 3 | 65.6 | 36.5 | P < 0.0001 | 44.4 | 39.1 |
| | 150 | 3 | 65.3 | 43.2 | P < 0.0001 | 33.8 | 44.1 |
| Commercial seals | Commercial seal a | 2 | 59 | 25 | P < 0.0001 | 66 | 16 |
| | Commercial seal b | 4 | 63 | 31 | P < 0.0001 | 51.4 | 13.6 |
| BTM-CCS study 1 | Seal based on the 150 μm | 12 | 64 | 31.4 | P < 0.0001 | 50.9 | 32.5 |
| Thickness and | Thin + holes | 2 | 66 | 57 | P > 0.05* | 13.7 | 69.9 |
| perforation | Thick no holes | 2 | 67 | 52 | P < 0.0001 | 22.6 | 73.9 |
| of seal | Thin no holes | 2 | 66 | 30 | P < 0.0001 | 54.8 | 84.2 |
| | Thick + holes | 2 | 66 | 30 | P < 0.0001 | 54.6 | 72.6 |
| BTM-CCS 2 | Seal based on new bond | 9 | 68.2 | 55.5 | P < 0.0001 | 18.6 | 40 |
| main study | | | | | | | |

BTM, biodegradable temporizing matrix;
CCS, cultured composite skin.
*A nonsignificant P value (P > 0.05) is a desirable result, signifying that the wound size has not significantly changed between initial creation and study completion.

(ii) The BTM-CCS Main Study Design

Animals

Three large domestic pigs (Sus scrofa) initially weighing 36 kg were acclimatised for one week prior to study commencement. Housing and animal care were provided in accordance with the local Animal Welfare Act and National Health and Medical Research Council (NHMRC) Guidelines. Sedation, anaesthesia and analgesia during surgery, dressing changes, and biopsy procedures were performed as previously described.

Biodegradable Polyurethane Matrices

BTM is a white, bioabsorbable polyurethane foam, sealed with a transparent non-absorbable polyurethane film. All BTM (sealed, 2 mm thick) and CCS matrices (unsealed, 1 mm thick) were sterile, dry-packed and provided by PolyNovo Biomaterials Pty. Ltd (Port Melbourne, Victoria, Australia).

Day 0: Skin Harvest and BTM Implantation

Four 8×8 cm study sites were designed (two on each flank of each animal). Thin split-thickness skin grafts were taken from each animal to provide the autologous components (fibroblasts and keratinocytes) for cell culture and composite construction. Blood (265 mls) from each animal was collected for the preparation of plasma and thrombin to aid in the generation of the composites.

One site remained untreated until the day of CCS application; the other three sites were then created to the level of the panniculus adiposus, ensuring that no dermal elements remained. A sealed, 2 mm thick BTM was then implanted into these deep wounds and held with surgical steel staples. All wounds were then dressed according to our established dressing protocol with Mepitel® (Mölnlycke, Gothenburg, Sweden) and Acticoat® (Smith & Nephew Ltd, Hull, UK) held with Hypafix (BSN Medical, Hamburg, Germany), overdressed with cotton combine also held with Hypafix; all protected by a custom-made pig coat. Wounds were assessed and dressed twice weekly until the CCS was ready for application on day 21.

Day 21—Fresh Wound Creation and CCS Application

The fourth site was excised to the same depth as the other three at day 21, and only received a cultured composite skin (CCS). At this same time point, the other three sites (where BTM had been implanted at day 0), were delaminated and the superficial surface abraded, ready to also receive a CCS. The composites were carefully applied, trimmed to size (if necessary) and affixed with surgical steel staples. A small Mepitel®, pre-cut to each individual treatment area, was affixed rostrally with staples to minimise shear trauma of the composites. The first dressing change allowed retention of this under-dressing. Standard dressing changes followed twice weekly.

CCS Generation (Day 0-21)

For CCS manufacture, cell isolation for keratinocytes and fibroblasts, plasma gel creation and cellular seeding were used as described above, with some minor modifications. These included use of a plasmin:thrombin ratio of 75:25 to provide a gel formation time of 20 to 30 seconds, and an increase in fibroblast seeding density (6×10⁴ cells per cm²) to enable a shorter fibroblast culture time in-matrix prior to co-culture, isolated cells were used from primary culture rather than passage 1 and the skin graft cell culture dishes (Nunc, N.Y., USA) included four inner chambers into which the matrices were pre-cut. The CCS was also ready for implantation on day 21, compared to day 28 in our previous study. One additional CCS was generated per pig for in vitro analysis.

Wound Assessment and Analysis

All research animals were anaesthetised with isofluorane with O₂ for wound evaluation and assessment. Wounds and peri-wound areas were gently cleaned with sterile saline before evaluation. Observations were recorded, photographs taken and wound areas measured using the Visitrak™ system (Smith & Nephew Ltd, Hull, UK) with evaporative water loss being assessed by a Vapometer (Delfin Technologies Ltd., Helsinki, Finland).

Throughout the study period, 4 mm punch biopsies were obtained for histological assessment from central wound locations. Large, full-thickness excision biopsies were collected at necropsy on day 52. All samples were fixed in 10% neutral-buffered formalin, dehydrated and embedded in paraffin. Sections were cut at 5 μm and stained with standard Haematoxylin and Eosin (H&E) and Periodic Acid-Schiff (PAS) stains. Some sections were rehydrated for immunohistochemical staining with antibodies for keratin (BovK; 1:500, Dako; Z0622). Immunofluorescence using viability dyes (Calcein/Ethidium, Sigma, St.Louis, Mo.) was also performed on biopsies taken from the additional CCS and viewed using the Nikon Confocal Microscope.

To compare the wound area and evaporative water loss between treatments and over time, a linear mixed effects model was fitted to the data. In the model treatment, time and the interaction between treatment and time were included as categorical predictor variables. Random effects for ‘pig’ and ‘pig within treatment’ were included in the model to account for the dependence in results due to repeated measurements on the same animal (multiple grafts and repeated measures over time). Post-hoc analysis was also performed within each treatment group from the original wound size to the final time point, to generate p-values (Table 1).

Post BTM-CCS Study: Optimisation of Seal Lamination/Bonding

A final seal optimisation study (aiming to demonstrate easy, quick, one-piece seal delamination) followed the BTM-CCS study. In this study, BTMs with a different seal lamination process were analysed in two pigs (8 treatment sites). All surgical procedures, dressing changes and wound measurement were performed as previously described.

Results

Optimisation of the Sealed Biodegradable Temporising Matrix (BTM)

The first groundwork study assessed seal type and thickness. Evidence of tissue in-growth and matrix integration was clearly visible and appears to occur remarkably early (7-10 days). This may be a major advantage over currently available two-stage dermal matrices that can take 2-4 weeks. The 50 μm and 100 μm seals were flexible and thus easy to apply, when compared to the stiffer 150 μm seal. However, these thinner seals displayed some micro-fracturing. Despite the 50 μm and 150 μm seals producing the largest wound areas at day 28 (45.9 cm² and 43.2 cm², respectively, Table 1), the 50 μm seal was omitted from further analysis because of microfragmentation and overgranulation through the resultant fissures. The 150 μm seal was the preferred seal in regards to seal retention.

A commercial seal (Tegaderm™, 3M Health Care, St Paul, Minn.) was then tested, bonded by two different techniques (labelled commercial a and b in Table 1). Although soft, supple and elastic, these became loosely detached by day 3 and completely lost by day 7. These were abandoned early because the spontaneous delamination resulted in minimal matrix integration and eventual loss of all foam matrices from all wound sites.

In a larger pig study, the first using CCS, BTM seals based on the 150 μm seal resulted in fragmentation and wrinkling which produced a sub-optimal wound bed for the application of the CCS. Although proof of concept was attained, with composite take being achieved, further seal refinements were obviously still required.

The next developmental stage was the assessment of whether perforated seals, to allow fluid drainage, would perform better than non-perforated seals, in laminae of varying thickness (50 or 150 μm). Neither thickness, nor the presence or absence of perforation alone, could be demonstrated to affect wound contraction (final wound size compared to initial wound size). Early delamination with unacceptable wound contraction was noted for both animals by day 14 for certain wounds where others maintained the BTM seals and resisted contraction. After unblinding, it was revealed that the manufacturers had used a different method of seal bonding for those matrices resulting in acceptable wound contraction (FIG. 6, seal types highlighted by oval) compared to those allowing unacceptable wound contraction.

Analysis of data from all the seal studies showed that final wound size correlated only with the BTM's ability to retain its seal, with early delamination allowing rapid wound contraction by study end. The post-hoc tests also indicated that most wound areas (regardless of seal type) had significantly contracted from their original wound size (p<0.0001, Table 1). From all of the optimisation studies there was only one seal, (thin+holes) that was not significantly different (p=0.685), i.e. there was no difference from starting wound size to end wound size (minimal contraction) (Table 1). FIG. 6 also demonstrates that the new bonding method employed for this seal was the contributing factor for maintaining final wound size, irrespective of seal thickness or perforation.

For evaporative water loss comparison, a high reading indicates a seal losing water, compared to a lower reading that indicates that the wound is physiological sealed. The evaporative water loss before delamination was not significantly different between all the different seals. All were, however, higher (Table 1) than normal skin (mean 7.8 g/m²h), but none displayed the very high readings noted from wounds left to heal by secondary intention, or those of unsealed wounds (generally >130 g/m²h). This suggests that maintenance of the seal, reducing evaporative water loss and thus resisting wound contraction, is pivotal to the success of the BTM. Thus, for the BTM-CCS main study, a sealed BTM based on the new bonding method was used.

BTM-CCS Main Study

Optimised BTM Ready for CCS Application

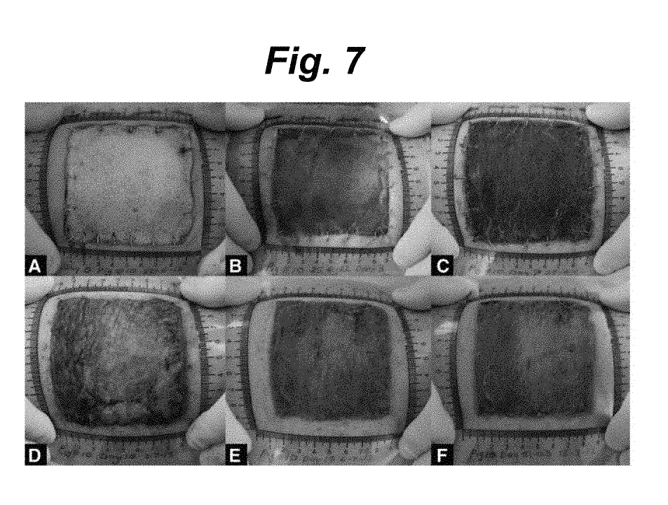

The bonding of the polyurethane seal to the matrix reduced early delamination, abolished fragmentation and restricted tissue in-growth to the matrix only. Whilst the seal was intact, the BTM performed its design function of integrating and resisting wound contraction whilst preparing the wound bed for definitive closure (FIG. 7). All BTMs integrated and maintained wound size (representative wound 65 cm² to 62.1 cm² with a 4.5% reduction in wound size, FIG. 7) with acceptable evaporative water loss over the 21 days (mean 40 g/m²h, Table 1). The integrated, sealed BTM was well vascularised, flush with the wound edge, soft and pliable, and clinically as thick as the surrounding skin. The seal was easy to delaminate by gentle teasing with a ‘Velcro-like’ action leaving vascularised tissue below. The seal showed no signs of spontaneous delamination. During surgical delamination, the superficial surface of the polymer separated (retracting back into tissue), leaving a partially refreshed wound bed, requiring minimal abrasion to refresh the surface. The abraded surface of the delaminated BTM bled gently, ready for CCS application.

Cultured Composite Skin (CCS)

CCS Before Application (In Vitro)

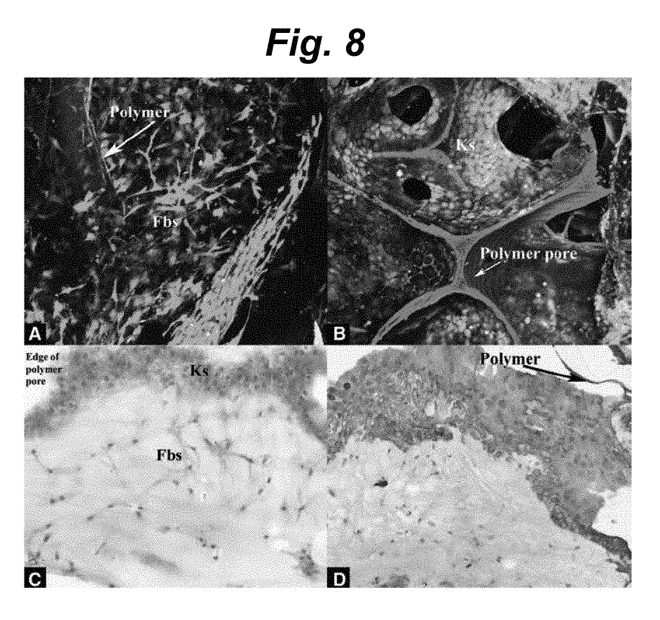

The fibroblasts showed typical bipolar morphology at both time points, the keratinocytes typically demonstrated a cuboidal, honeycomb appearance (FIG. 3). Histological sections and immunohistochemical staining demonstrated that the walls of the pores become lined with cells (FIG. 8). However, cell distribution was not uniform and appeared to be sporadic i.e. some pores demonstrating very few cells. By day 21 of in-matrix culture, the majority of keratinocytes were identified in the deeper levels of the CCS (within the lower 200-300 μm of the 1 mm composite).

CCS Post-Application on BTM-Implanted Wounds (In Vivo)

Once the CCS had been applied to the temporised wound bed, two phenomena were noted, both of which had been observed in previous studies.

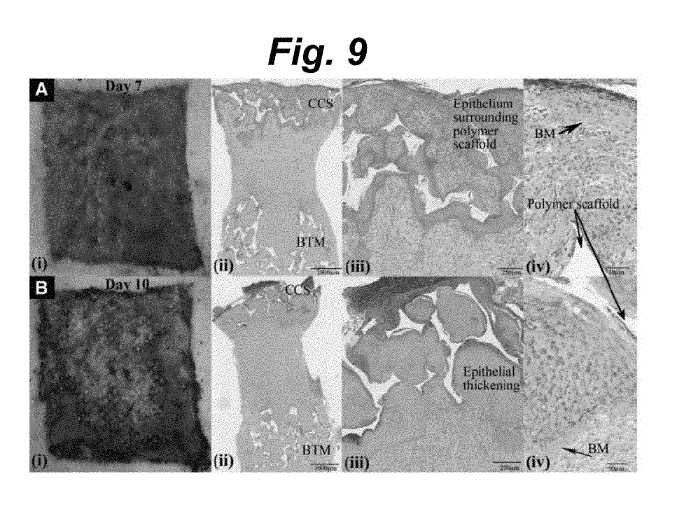

i). CCS take: ‘Take’ denotes that both matrix and cellular components are retained as part of the healed tissue. The degree of take varied within BTM-CCS treated wounds, in general this was observed early (by day 7 post-application) (FIG. 9Ai-iv). By day 10, most of the polymer was evident in the hyperkeratotic layer external to a well-developed epidermis anchored by a basement membrane (FIG. 9 Bi-iv). In some sections, polymer microfragments were embedded deep in dermal invaginations of epidermis. By the study end-point at day 31 post-CCS application (day 52 from original wounding), there were sections of CCS that had been fully incorporated into the wound, however the majority of the CCS foam had been shed, due to epidermal sloughing, but it was left with a robust, stratified epithelium with a well-developed basement membrane. Evaporative water loss from this wound at day 31 was relatively low (34.9 g/m²h), suggesting epithelial closure and stratum corneum development (Table 2).

TABLE 2
Representative evaporative water
loss from the BTM-CCS main study

| | Evaporative Water Loss (g/m²h) | |
|---|---|---|
| BTM-CCS Main Study | Postapplication or Wound Generation | |
| Wound Type | Day 14 | Day 31 |
| Fresh wound generated with CCS | 39.4 | 22.7 |
| only (no BTM) | | |
| BTM-CCS take | 46 | 34.9 |
| BTM-CCS delivery vehicle | 20.6 | 10.1 |
| Normal skin | 4.9 | 5.4 |
| Wound left to heal by secondary | 139 | 26.3* |
| intention | | |

BTM, biodegradeable temporizing matrix; CCS, cultured composite skin.
*By day 31 after generation, the secondary intention wound had healed.

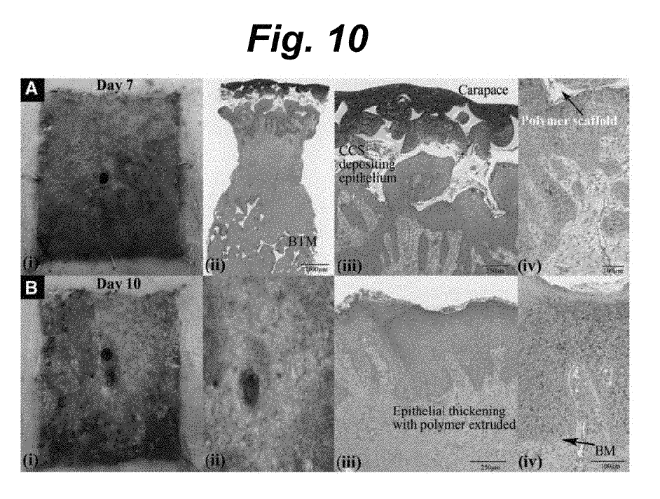

ii). CCS as a cellular delivery vehicle (FIG. 10Ai-iv): In the first few days after application, it appeared to develop a thin ‘carapace’ over the wound (FIG. 10Aiii). This structure could be removed easily at day 10. Upon removal, we discovered a fully formed epithelium had developed on the underlying wound surface. This was confirmed with punch biopsies on day 10 that showed a well developed epithelium and basement membrane (FIG. 10Bi-iv). This leads us to surmise that the CCS delivers its cellular content and then persists (like a scab) until encouraged to separate by stratum corneum shedding from the underlying formed epidermis. In this situation therefore, the wound was virtually healed 10 days after CCS application. A decline in evaporative water loss readings also indicated wound closure and epithelium thickening over time, with a reading of 20.6 g/m²h on day 14 and by day 31 post-application it was 10.1 g/m²h, close to that of normal skin (Table 2).

CCS Only (no BTM)

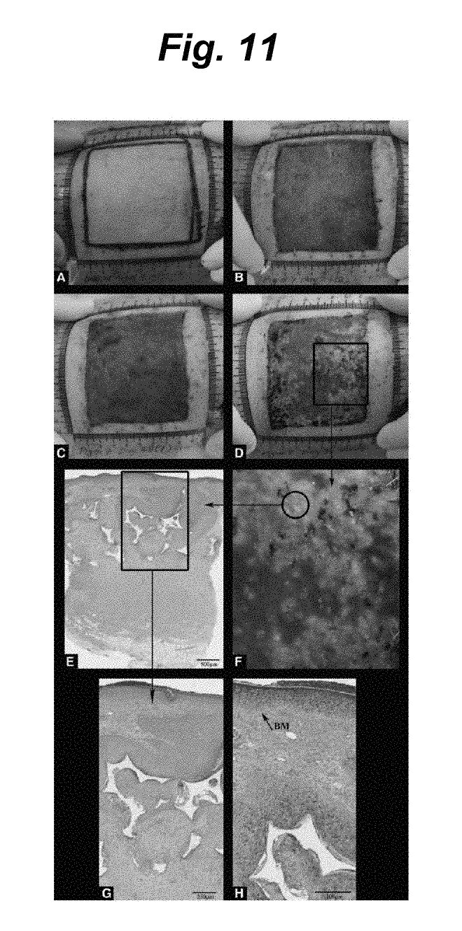

CCS retention in the fresh wounds (no BTM) was observed in two out of three wounds. Within the first three days of application, one CCS treatment area was completely lost due to animal trauma, allowing it heal by secondary intention. There were no residual polymer remnants.

Where the CCS was implanted into the fresh wounds created at day 21, the clinical course appeared different (FIG. 11). By day 7 post-CCS implantation, no clinical composite foam was visible, the CCS had completely integrated within exuberant vascular tissue filling the wound to the flush point (FIG. 11c). The beginning of epithelialisation was evident at day 10 with individual islands/foci of epithelium visible (FIG. 11d-e). A punch biopsy from one of these foci showed CCS incorporation with developing epithelium and basement membrane formation (FIG. 11f-h). The epithelial foci became more complete as time progressed and were visible in all areas of the wound simultaneously, demonstrating that the origin of these epidermal islands was the CCS, not the wound edge. By day 31 post-application (day 52, study end-point), the CCS scaffold was completely integrated, with the majority disposed in the deep dermis abutting upon subcutaneous fat. Clinically, the wound was almost completely re-epithelialised (˜95%), with a stratified squamous epithelium with continuous basement membrane and rete pegs. The evaporative water loss from this wound at day 31 was also indicative of wound closure (22.7 g/m²h, Table 2).

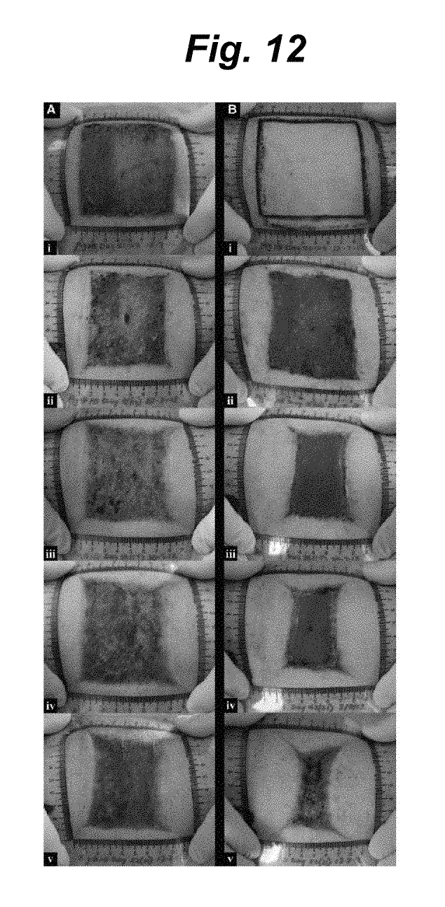

This was very different from the pattern of re-epithelialisation which progresses from the wound margins following early severe wound contraction in the wound allowed to heal by secondary intention (FIG. 12Bi-v). By day 31 post wound generation, this wound had reduced from its original size by 64%, compared to a wound that healed through the application of a BTM followed by CCS (with only a 19% reduction by day 52 after original wound creation). Although the healed wound with CCS remained visually pink to study end, and clinically might not appear closed on photographs, histologically by day 17, the BTM-CCS had completely re-epithelialised, with further epithelial thickening by study end-point (day 31 post-application) (FIG. 12Ai-iv). A completely healed wound with colour akin to neighbouring skin might have been more convincing, but time constraints and housing costs for maintaining pigs for an extended period (beyond the 52 day time point) was not feasible in these studies.

Previously we have shown that a sub-optimal integrated BTM wound bed will support CCS take. Here, we show that the CCS was demonstrably capable of generating a bilayer repair over both the abraded surface of the fully integrated BTM, and the fresh wound's fat base. The mode of action of the CCS i.e. take or cellular delivery appeared to depend on the deposition of the cells within the composite. Regardless, wound closure needs to occur within the first 7-10 days post-application to ensure that the granulation layer generated between CCS and integrated BTM is kept to a minimum, in turn determining the degree of contraction.

Post BTM-CCS Study: Seal Lamination/Bonding Optimised

The pilot human BTM trial (that used the same BTM as the BTM-CCS main study) revealed that unlike in the porcine model, the seal tore upon delamination and had to be removed in more than one action. This made the removal process unacceptably time-consuming, warranting further seal structure/bond optimisation. Therefore, another study was performed to assess a new BTM seal which had been designed to resolve the issues with seal fragmentation upon delamination.

The new materials were easy to cut and affix and felt slightly more robust than previous iterations. By day 3 post-implantation, all matrices were coloured by vascular fluid, but lacked the solid appearance of tissue ingrowth. The seals showed no tearing at staple sites, fragmentation or signs of early delamination. By day 7, the BTM colour was much darker with obscurity of the polymer foam structure indicating solidity of the contents and tissue integration. Some wound contraction was evident in the shoulder treatment areas, typically seen in this model. Three treatment sites were removed from analysis due to repeated trauma (at both flank and shoulder areas) causing the seal to delaminate from day 21, such that they could not be included in determining the final mean. By day 28, the largest wound area was 55.3 cm², a 12% reduction from its original size (63.5 cm²). The mean wound area (n=5) for the new seals was 64.3 cm² after creation and by day 28 it was 53 cm² (18% contraction). This is lower than those reported in a study that compared a number of commercially-available dermal matrices also in a porcine model (Integra-25%, Hyalomatrix-24%, Matriderm-28%, Renoskin-34%) 18 Although a direct comparison of BTM against other dermal matrices was not performed, our data showed that the wound contraction rate of BTM was comparable to a split-thickness graft applied at the same time-point,13 and was significantly lower than a secondary intention healing wound (up to 55% reduction in wound area over a 21 day period 13).

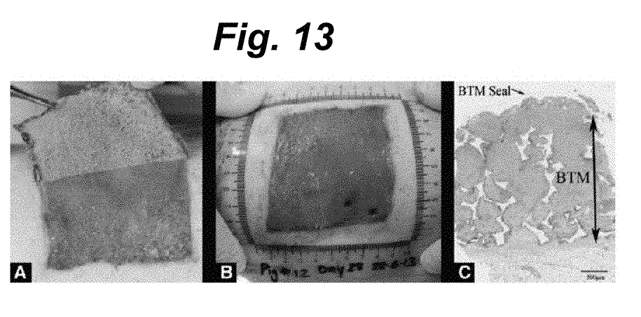

Upon delamination at day 28, the seal was removed easily and rapidly in one single piece and action, leaving behind a highly vascularised and clean wound bed (FIG. 13a-b). Histological analysis of the punch biopsy prior to delamination revealed seal/matrix adhesion with no significant development of a scar layer between the seal and the polymer matrix. The BTMs were closely apposed to the fat in the original wound base (FIG. 13c). Evaporative water loss from the sealed matrices over the period of the study ranged between 10-17 g/m²h (mean 13.84 g/m²h), with normal skin averaging 4.4 g/m²h. This outperformed the previous best BTM seal (40 g/m²h) and demonstrated that the seals had successfully mimicked wound closure. Furthermore, the results of the evaporative water loss from the optimised sealed BTM was also similar to a split-thickness graft 13 and clearly less than a wound allowed to heal by secondary intention or an unsealed matrix. Demonstrating the suitability of BTM as a dermal substitute.

### EXAMPLE 10

### A Bioreactor System for Producing Composite Skin

A cell or tissue culture system (typically referred to as a “bioreactor”) may be used to produce a cultured skin product, for example for the treatment of burns or wounds. Bioreactors are known in the art, for example as described in Hambor J. (2012) BioProcess International Volume 10(6).

Cells may be incorporated in a porous, biodegradable polyurethane foam, as described herein. Sheets of foam of a size of 25×25 cm² may be used to form a gel with cells in situ in the foam in a culture plate. As described herein, autologous fibroblast cells may first be incorporated in the foam. After setting, the foam with incorporated cells in a culture plate may be transferred to a perfusion bioreactor and a suitable medium (for example DMEM-10% FBS) pumped in at a suitable flow rate (for example 5 ml/minute).

After culturing of the fibroblast cells, the keratinocytes may subsequently be incorporated into the porous matrix, and following setting of the gel, the culture plate transferred back to the bioreactor and medium (SEL-KGM 5% FBS) pumped in at a suitable flow rate (for example 5 ml/minute).

The matrix with incorporated cells may then be incubated in the bioreactor system until the composite culture skin is suitable for transplantation. Culture medium may be replaced in the bioreactor system, for example every 2 to 3 days.

### EXAMPLE 11

### Kits

Kits for use in the methods of the present disclosure are contemplated.

A kit may include one or more of the following components:

(a) A matrix for temporising a wound. For example, a 2-mm thick foam, biodegradable matrix produced from a polyurethane with a 150-μm thick, nonbiodegradable, microporous polyurethane sealing membrane bonded to the surface.

(ii) Reagents for isolating cells from skin biopsies, such as antibiotics, PBS, Dispase II, trypsin-EDTA, trypsin inhibitor, culture medium, supplements and Colleganase I.

(iii) A porous matrix for incorporating cells, such as 25×25 cm² sheets of biodegradable, 1 mm thick porous polyurethane fibrous matrix, typically γ-irradiation sterilised and held in sealed packages.

(iv) Thrombin.

(v) Tissue culture plates for receiving sheets of foam, typically sterilised and packaged.

(vi) Reagents for incubating a porous matrix to expand cells, such as fibroblast culture medium and/or keratinocyte culture medium, with supplements.

(vii) Instructions.

One or more of the kit components of the kit may be packaged in sterile form.

Although the present disclosure has been described with reference to particular examples, it will be appreciated by those skilled in the art that the disclosure may be embodied in many other forms.

Reference to any prior art in this specification is not, and should not be taken as, an acknowledgment or any form of suggestion that this prior art forms part of the common general knowledge in any country.

As used herein, the singular forms “a,” “an,” and “the” may refer to plural articles (i.e., “one or more,” “at least one,” etc.) unless specifically stated otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The subject headings used herein are included only for the ease of reference of the reader and should not be used to limit the subject matter found throughout the disclosure or the claims. The subject headings should not be used in construing the scope of the claims or the claim limitations.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of incorporating cells in a porous matrix, the method comprising incorporating the cells in the porous matrix by forming a gel comprising the cells in situ in the porous matrix from a gellable medium, wherein the formation of the gel from the gellable medium comprises a time period of 10 minutes or less after contact of the gellable medium with the cells, thereby incorporating the cells in the porous matrix,
wherein the method comprises one or more rounds of gel formation incorporating cells in the porous matrix, one or more rounds of applying fibroblast cells or a progenitor thereof, or both, to form a gel comprising fibroblast cells or a progenitor thereof, or both, and one or more rounds of applying keratinocyte cells, or a progenitor thereof, or both, to form a gel comprising keratinocytes cells,
wherein the keratinocyte cells are applied subsequent to the applying of fibroblast cells.

2. The method according to claim 1, wherein the formation of the gel comprises a time period of 10 to 300 seconds after contact of the gellable medium with the cells.

3. The method according to claim 1, wherein the formation of the gel comprises a time period of one of 10 to 50, 20 to 50, 25 to 50, 30 to 50, 10 to 30, or 20 to 30 seconds after contact of the gellable medium with the cells.

4. (canceled)

5. The method according to claim 1, wherein the porous matrix comprises one or more of a polyurethane, a collagen, a glycosaminoglycan, a collagen-GAG co-polymer and/or mixture, a mucopolysaccharide, a poly(alpha-hydroxy acid), a poly(lactic acid) or a structural isomer thereof, a polyglycolic acid or a structural isomer thereof, a chitosan, a polylactic-polyglycolic polymer, and a polycaprolactone.

6. The method according to claim 1, wherein the porous matrix is biodegradable in vivo.

7. The method according to claim 1, wherein the porous matrix comprises an average pore size in the range from 100 μm to 1000 μm.

8. The method according to claim 1, wherein the porous matrix comprises an average pore size in the range from 200 μm to 500 μm.

9. The method according to claim 1, wherein the porous matrix comprises a fibrous porous matrix.

10. The method according to claim 9, wherein the fibrous porous matrix comprises fibres with a diameter in the range from 50 μm to 200 μm.

11. The method according to claim 9, wherein the fibrous porous matrix comprises fibres with a diameter in the range from 60 μm to 100 μm.

12. The method according to claim 1 wherein the porous matrix is in the form of a sheet.

13. The method according to claim 12, wherein the sheet comprises a thickness in the range from 1 mm to 3 mm.

14. The method according to claim 12, wherein the cells applied to the porous matrix comprise 1×10⁴ to 1×10⁵ cells per cm² of the porous matrix.

15. The method according to claim 12, wherein the cells applied to the porous matrix comprise 4×10⁴ or greater cells per cm² of the porous matrix.

16. The method according to claim 1, wherein the method comprises applying the cells and the gellable medium to the porous matrix at the same time.

17. The method according to claim 1, wherein the method comprises applying the gellable medium to the porous matrix and subsequently applying the cells to the porous matrix.

18. The method according to claim 1, wherein the method comprises use of a gelling agent to form a gel from the gellable medium.

19. The method according to claim 18, wherein the cells and the gelling agent are applied to the porous matrix at the same time.

20. The method according to claim 18, wherein the method comprises applying the gellable medium to the porous matrix, and subsequently applying cells and a gelling agent to the porous matrix .

21. The method according to claim 1, wherein the gellable medium comprises plasma and/or a derivative thereof.

22. The method according to claim 21, wherein the derivative comprises fibrinogen.

23. The method according to claim 18, wherein the gelling agent comprises thrombin.

24. The method according to claim 23, wherein the use of the thrombin comprises a plasma/thrombin ratio of 60:40 to 85:15.

25. (canceled)

26. (canceled)

27. (canceled)

28. The method according to claim 1, wherein the method further comprises incubating the cells incorporated in the porous matrix to expand cell number.

29. The method according to claim 1, wherein the method comprises use of a bioreactor.

30. (canceled)

31. (canceled)

32. The method according to claim 1, wherein the method is used to produce a product for producing skin, to produce a cultured skin product, to deliver cells to a subject, or to deliver one or more cell derived factors to a subject.

33. (canceled)

34. (canceled)

35. (canceled)

36. (canceled)

37. (canceled)

38. (canceled)

39. (canceled)

40. A method of producing a product for forming skin, the method comprising:
applying plasma to a biodegradable porous matrix;
applying fibroblast cells, or a progenitor thereof, or both, and thrombin to the biodegradable porous matrix with applied plasma to form a gel comprising fibroblast cells incorporated in the porous matrix;
incubating the porous matrix with incorporated fibroblast cells;
applying keratinocyte cells, or a progenitor thereof, or both, and thrombin to the porous matrix with incorporated fibroblast cells to form a gel comprising keratinocyte cells incorporated in the porous matrix; and
incubating the porous matrix with incorporated fibroblast cells and keratinocyte cells;
wherein the formation of the said gels comprises a time period of 10 minutes or less after contact of the plasma with the cells;
thereby producing a product for forming skin.

41. (canceled)

42. (canceled)

43. A method according to claim 40, further comprising introducing the product for forming skin in a subject, whereby the skin is formed in the subject.

44. The method according to claim 43, wherein the cells comprise autologous cells.

45. A method according to claim 40, further comprising applying the product for forming skin to a wound in the subject, thereby treating the wound.

46. The method according to claim 45, wherein the wound comprises a burn, a post-operative wound, a chronic wound, or a diabetic ulcer.

47. A method of producing a product for forming skin in a subject, the method comprising:
applying autologous plasma, or a derivative thereof, or both, to a biodegradable porous matrix;
applying autologous fibroblast cells, or a progenitor thereof, or both, and thrombin to the biodegradable porous matrix with applied plasma to form a gel comprising fibroblast cells incorporated in the porous matrix;
incubating the porous matrix with incorporated fibroblast cells;
applying autologous keratinocyte cells, or a progenitor thereof, or both, and thrombin to the porous matrix with incorporated fibroblast cells to form a gel comprising keratinocyte cells incorporated in the porous matrix; and
incubating the porous matrix with incorporated fibroblast cells and keratinocyte cells;
wherein the formation of the said gels comprises a time period of 10 minutes or less after contact of the plasma with the cells;
thereby producing a product for forming skin in a subject.

48. A method of treating a wound in a subject, the method comprising:
applying a seal to the wound to close the wound or reduce evaporative water from the wound, or both;
removing the seal from the wound; and
applying the product for forming skin produced according to 47 to the wound;
thereby treating the wound.

49. (canceled)

50. (canceled)

51. (canceled)

52. (canceled)

53. (canceled)
